(19) United States
(12) Patent Application Publication
(10) Pub. No.: US 2023/0390171 A1
(43) Pub. Date: Dec. 7, 2023

(54) DEODORANT COMPOSITIONS COMPRISING LIPOPHILIC CARBOXYLIC ACIDS

(71) Applicant: The Procter & Gamble Company, Cincinnati, OH (US)

(72) Inventors: Jonathan Robert Cetti, Mason, OH (US); Ryan Michael West, West Chester, OH (US); Kevin Robert Johnstone, Cincinnati, OH (US); Sebastian Karol Galazka, Loveland, OH (US); Brooke Michele Cochran, Cincinnati, OH (US)

(21) Appl. No.: 18/327,175

(22) Filed: Jun. 1, 2023

Related U.S. Application Data
(60) Provisional application No. 63/348,086, filed on Jun. 2, 2022.

(51) Int. Cl. A61K 8/36; A61Q 15/00; A61K 8/33; A61K 8/49; A61K 8/891; A61K 8/06; A61K 8/04; A61K 8/368; A61Q 17/00

(52) U.S. Cl. CPC A61K 8/361; A61Q 15/00; A61K 8/33; A61K 8/4926; A61K 8/891; A61K 8/064; A61K 8/042; A61K 8/368; A61Q 17/005

(57) ABSTRACT

A deodorant composition comprising a lipophilic carboxylic acid, wherein the lipophilic carboxylic acid has a C Log D from −0.5 to 3 at a pH from 3 to 5, and at least about 20%, by weight of the composition, of a short-chain glycol, wherein the composition is free of aluminum.

### FIELD OF THE INVENTION

The present disclosure relates to deodorant compositions and methods relating thereto.

### BACKGROUND OF THE INVENTION

One of the main functions of a deodorant or antiperspirant product is to control unpleasant body odor. At least some body odor is the result of microorganisms on the skin that break down sweat to produce the smell that is associated with body odor. Thus, there is a need for deodorant and antiperspirant compositions that neutralize body odor by preventing the bacteria that create it. While many antimicrobials are known to formulators, not just any antimicrobial is easily incorporated into a deodorant or antiperspirant product in any product form. Additionally, while aluminum has been used for many years as an effective odor reducer by reducing perspiration, there is consumer interest in antiperspirants and deodorants that do not contain aluminum.

Additionally, many consumers seek more natural antiperspirants and deodorants, for example, ones that are silicone-free. Thus, there is a continuing need and challenge to formulate aluminum-free and natural deodorants that effectively offer odor protection.

### SUMMARY OF THE INVENTION

A deodorant composition comprising:
a. a lipophilic carboxylic acid, wherein the lipophilic carboxylic acid has a C Log D from −0.5 to 3 at a pH from 3 to 5; and
b. at least about 20%, by weight of the composition, of a short-chain glycol;
wherein the composition is free of aluminum.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 is a graph of the C log D of various carboxylic acids at various pH's.

FIG. 2 shows proper swabbing for the Finished Product Soleris Method.

### DETAILED DESCRIPTION OF THE INVENTION

While the specification concludes with claims that particularly point out and distinctly claim the invention, it is believed the present invention will be better understood from the following description.

The present invention can comprise, consist of, or consist essentially of the essential elements and limitations of the invention described herein, as well any of the additional or optional ingredients, components, or limitations described herein.

All percentages, parts and ratios are based upon the total weight of the compositions of the present invention, unless otherwise specified. All such weights as they pertain to listed ingredients are based on the active level and, therefore do not include carriers or by-products that may be included in commercially available materials.

The components and/or steps, including those which may optionally be added, of the various embodiments of the present invention, are described in detail below.

All documents cited are, in relevant part, incorporated herein by reference; the citation of any document is not to be construed as an admission that it is prior art with respect to the present invention.

All ratios are weight ratios unless specifically stated otherwise.

All temperatures are in degrees Celsius, unless specifically stated otherwise.

Except as otherwise noted, all amounts including quantities, percentages, portions, and proportions, are understood to be modified by the word “about”, and amounts are not intended to indicate significant digits.

Except as otherwise noted, the articles “a”, “an”, and “the” mean “one or more”.

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

Herein, “effective” means an amount of a subject active high enough to provide a significant positive modification of the condition to be treated. An effective amount of the subject active will vary with the particular condition being treated, the severity of the condition, the duration of the treatment, the nature of concurrent treatment, and like factors.

The term “anhydrous” as used herein means substantially free of added or free water. From a formulation standpoint, this means that the anhydrous deodorant stick compositions of the present invention contain less than about 1%, and more specifically zero percent, by weight of free or added water, other than the water of hydration typically associated with the particulate deodorant active prior to formulation.

The term “ambient conditions” as used herein refers to surrounding conditions under about one atmosphere of pressure, at about 50% relative humidity, and at about 25° C., unless otherwise specified. All values, amounts, and measurements described herein are obtained under ambient conditions unless otherwise specified.

The term “majority” refers to greater than about 51% of the stated component or parameter.

“Substantially free of” refers to about 2% or less, about 1% or less, or about 0.1% or less of a stated ingredient. “Free of” refers to no detectable amount of the stated ingredient or thing.

The term “volatile” as used herein refers to those materials that have a measurable vapor pressure at 25° C. Such vapor pressures typically range from about 0.01 millimeters of Mercury (mm Hg) to about 6 mmHg, more typically from about 0.02 mmHg to about 1.5 mmHg; and have an average boiling point at one (1) atmosphere of pressure of less than about 250° C., more typically less than about 235° C. Conversely, the term “non-volatile” refers to those materials that are not “volatile” as defined herein.

### Lipophilic Carboxylic Acids

Carboxylic acids are a class of materials that have been used in the cosmetic industry and are effective in providing anti-aging and moisture retention benefits to the skin. Some carboxylic acids are known to be effective antimicrobials. Carboxylic acids can be linear or branched, saturated or unsaturated, or contain additional hydroxy groups beyond the carboxylic acid moiety. Monocarboxylic acids can be defined as having a single carboxylic acid moiety with any of the preceding characteristics. Dicarboxylic acids can be defined as having two carboxylic acid moieties on a molecule with any of the preceding characteristics.

Alpha hydroxy acids are a special class of carboxylic acids that have long been used in the cosmetics industry. Some alpha hydroxy acids, such as mandelic acid, are known to be effective antimicrobials. Knowing this, the present inventors screened various alpha and beta hydroxy acids in a simplified aluminum-free deodorant formulation for effectiveness against some common bacteria known to cause body odor. Certain alpha hydroxy acids performed better than others. The initial hypothesis was that a more robust ability to prevent bacteria propagation was driven by the pKa of the acid. But while the pKa was important, the present inventors have discovered that the driving factor is primarily related to the lipophilicity, specifically the C log D of the material. C log D is the pH-dependent octanol:water partition coefficient for ionizable compounds.

The present inventors have discovered that an unexpected bacteria-preventing performance occurs with lipophilic carboxylic acids, defined as those that have a C log D of −0.5 to 3 at pH 3.0 to 5.0. While not being bound by theory, the inventors believe that at pH values of around 3.5-4.5, the carboxylic acids prefer to partition into an octanol phase from water with an equal preference at a value of 0, and 1000:1 preference at a value of 3. This range appears to represent a range that especially inhibits bacterial growth. Materials that are too lipophilic likely phase separate from the formulation and are not able to be effectively solubilized in human perspiration. Other materials may be not lipophilic enough, such that they do not have a driving force to interact with the lipophilic bacteria membrane. In other words, the carboxylic acids within the identified ranges may be able to prevent bacterial growth, thus reducing the formation of body odor. The pH-Dependent Octanol-Water Partition Coefficient (log D) is defined as the equilibrium distribution between a non-polar octanol phase and a polar aqueous phase of all solute species present at a given pH (of the aqueous phase) at 25° C. It is computed in this instance using the ACD/Labs Log D module. For compounds with ionizable functional groups (acids and bases), the partition coefficient calculation takes the apparent ionization state of each ionizable group into account through the calculation of the pKa for each functional group. The partition coefficient is then computed at different solution pH values and reported. In this instance, the log D of a solute is computed over the range from pH=2.0 to pH=12.0, in steps of 0.5 pH units.

The lipophilic carboxylic acids in the present invention may include mandelic acid, hydroxycapric acid, azelaic acid, salicylic acid, or combinations thereof. The carboxylic acid's total number of carbons may be from C7 to C11.

This mode of action for prevention of bacterial growth by carboxylic acids may also be pH dependent, thus the formulation pH is important. Maintaining a pH near the pKa or lower will keep the acid protonated, which improves ingredient potential for control of bacteria. For a consumer-preferred feel on the skin, there is also a lower limit for the pH to stay above. Formulations must balance these two factors.

Also important to an effective formulation may be the inclusion of an additional antimicrobial. The inclusion of certain carboxylic acids alone provides good prevention of bacterial growth, but in order to achieve an even higher level of bacterial growth inhibition and corresponding odor protection, an additional antimicrobial may be used. For example, additional antimicrobials may include, without being limited to, hexamidine, thymol, polyvinyl formate, niacinamide, cinnamon essential oil, cinnamon bark essential oil, cinnamic aldehyde, piroctone olamine, octenidine dihydrochloride, polydialyldimethylammonium chloride, polyquaternium, and combinations thereof. In some embodiments, the antimicrobial may be a quaternary antimicrobial such as octenidine dihydrochorloide or polyquaternium.

In certain embodiments, the inventors of the present invention believe that octenidine dihydrochloride or piroctone olamine may be good antimicrobials to combine with lipophilic carboxylic acids. Without being bound by theory, the present inventors believe that the lipophilic carboxylic acids of the claimed invention can disrupt the lipophilic wall of the cell membrane, thus enabling the antimicrobial to penetrate the cell wall more effectively and lessen the propagation of bacteria growth. While previous studies have shown that quaternary antimicrobials will have a higher degree of hostility at an alkaline pH (See “pH Influence on Antibacterial Efficacy of Common Antiseptic Substances”, by Cornelia Wiegand, Martin Abel, Peter Ruth, Peter Elsner, and Uta-Christina Hipler, published Jan. 20, 2015 in Skin Pharmacology and Physiology, Skin Pharmacol Physiol 2015; 28:147-158), the present inventors have unexpectedly discovered that some antimicrobials can contribute to a higher degree of hostility at a lower pH when combined with the lipophilic carboxylic acids of the claimed invention. (See tables 6 and 7 for further discussion).

### Glycol

Deodorant formulations may optionally contain glycols. When used as a carrier, glycols are known in the art to promote a hostile environment for bacterial growth. Glycol materials may include, but are not limited to, dipropylene glycol, propylene glycol, 1,3 Propanediol, butylene glycol, tripropylene glycol, hexylene glycol, 1,2 hexane diol, PPG-10 butanediol, and polyethylene glycol. Glycols may be particularly useful for aqueous composition, to provide solvency for lipophilic materials such as lipophilic carboxylic acids and antimicrobials. For compositions containing silicone or triglycerides as a carrier, glycols may not be needed to solubilize lipophilic carboxylic acids and antimicrobials. In such compositions, glycols may disrupt the creation of a solid stick by inhibiting the proper crystallization of the wax structurants due to the high degree of polarity coming from the short chain glycols, thus not achieving the desired hardness.

Dipropylene glycol and propylene glycol are both known in the art to promote a hostile environment to bacterial growth. The inventors of the present invention believe that dipropylene glycol is a good carrier to combine with other antimicrobials. Deodorant compositions may comprise from about 0% to about 65%, from about 0% to about 50%, from about 10% to about 55%, from about 10% to about 50%, from about 20% to about 50%, from about 30% to about 50%, or from about 30% to about 55%, of any glycol disclosed herein, but dipropylene glycol or propylene glycol in particular, by weight of the composition. The glycols used in the present invention may be a single or a combination of short-chain glycols, or a combination of short-chain and longer-chain glycols. A short-chain glycol means comprising at most a C12 chain length, in some embodiments at most a C11, C10, or C9 chain length.

Glycols may be used in clear gel deodorant compositions that may be water-in-oil emulsions. The glycols may be used to adjust the refractive index of the water phase so that it matches the refractive index of the oil phase (preferably to within about 0.0004) in order to achieve maximum clarity of the final composition. For optimum clarity the refractive index of the oil phase and the water phase should be matched to within about 0.001 or better, preferably to within about 0.0004.

Deodorant formulations may optionally contain a polyether compound. Polyether compounds may include, but are not limited to, polyethylene glycols and polypropylene glycols. Polyether compounds suitable for use in the deodorant compositions include, but are not limited to, PEG-4 (also called PEG 200 or polyethylene glycol with average molecular weight of 200 daltons), PEG-6 (also called PEG 300 or polyethylene glycol with average molecular weight of 300 daltons), PEG-8 (also called PEG 400 or polyethylene glycol with average molecular weight of 400 daltons), PEG-12 (also called PEG 600 or polyethylene glycol with average molecular weight of 600 daltons), polypropylene glycol (like dipropylene glycol, tripropylene glycol, PPG-3, PPG-6, PPG-9, PPG-12, PPG-15, etc.), diethylene glycol, triethylene glycol, and combinations thereof.

Polyether compounds may be particularly useful for aqueous compositions to provide solvency for lipophilic materials such as lipophilic carboxylic acids and antimicrobials. For clear gel deodorant compositions, polyether compounds are particularly useful as these materials have a higher refractive index than glycols and therefore are more effective at adjusting the refractive index of the water (also referred to as polar or aqueous) phase to the oil (also referred to as nonpolar or silicone) phase in order to achieve maximum clarity of the final composition. Deodorant compositions may comprise from about 0% to about 65%, from about 0% to about 50%, from about 10% to about 55%, from about 10% to about 50%, from about 20% to about 50%, from about 30% to about 50%, from about 30% to about 55%, from about 20% to about 45%, or from about 25% to about 40% of any polyether compound disclosed herein by weight of the composition.

The gel composition m a y have a clarity better than 100 NTU (Nephelometric Turbidity Units), preferably better than 75 NTU, and most preferably better than 50 NTU at 21° C.

In some embodiments, there may be a water phase and a silicone phase. And in some embodiments, the refractive index of the water phase may be the same as the silicone phase, to within about 0.001 or better, preferably to within about 0.0004.

In some embodiments, the composition may have a percent transmittance (% T) of at least about 80% transmittance at 600 nm. In some embodiments, the refractive index of the water phase may be from 1.3500 to 1.4300.

In some embodiments, the composition may comprise at least 3% ethanol, by weight of the composition, or at least 0.25% of an ionic salt, by weight of the composition, and in some embodiments, the ionic salt may be sodium chloride.

### Deodorant Compositions

The deodorant compositions of the present invention may take one of many forms. Inventive forms may include a roll-on, solid sticks, (clear) gels, soft solid, sprays, cream, lotion, or serum. Below are lists of materials for various forms of the deodorant compositions. A roll-on deodorant composition can comprise, for example, water, emollient, solubilizer, deodorant actives, antioxidants, preservatives, or combinations thereof. A clear gel deodorant composition can comprise, for example, water, emollient, solubilizer, deodorant actives, antioxidants, preservatives, ethanol, or combinations thereof. A solid stick deodorant composition can comprise, for example emollient, deodorant actives, waxes, or combinations thereof.

Water

The deodorant composition can include water. Water can be present in an amount of about 1% to about 99.5%, about 1% to about 30%, about 1% to about 50%, about 10% to about 30%, about 25% to about 99.5%, about 50% to about 95%, about 50% to about 99.5%, about 75% to about 99.5% about 80% to about 99.5%, from about 15% to about 45%, or any combination of the end points and points encompassed within the ranges, by weight of the deodorant composition.

Emollients

The deodorant composition can comprise an emollient system including at least one emollient, but it could also be a combination of emollients. Suitable emollients are often liquid under ambient conditions. Depending on the type of product form desired, concentrations of the emollient(s) in the deodorant compositions can range from about 1% to about 95%, from about 5% to about 95%, from about 15% to about 75%, from about 1% to about 10%, from about 15% to about 45%, or from about 1% to about 30%, by weight of the deodorant composition.

Emollients suitable for use in the deodorant compositions include, but are not limited to, propylene glycol, polypropylene glycol (like dipropylene glycol, tripropylene glycol, etc.), diethylene glycol, triethylene glycol, PEG-4, PEG-8, 1,2 pentanediol, 1,2 hexanediol, hexylene glycol, glycerin, C2 to C20 monohydric alcohols, C2 to C40 dihydric or polyhydric alcohols, alkyl ethers of polyhydric and monohydric alcohols, volatile silicone emollients such as cyclopentasiloxane, nonvolatile silicone emollients such as dimethicone, mineral oils, polydecenes, petrolatum, and combinations thereof. One example of a suitable emollient comprises PPG-15 stearyl ether. Other examples of suitable emollients include dipropylene glycol and propylene glycol.

Deodorant Actives

Suitable deodorant actives can include any topical material that is known or otherwise effective in preventing or eliminating malodor associated with perspiration. Suitable deodorant actives may be selected from the group consisting of antimicrobial agents (e.g., bacteriocides, fungicides), malodor-absorbing material, and combinations thereof. For example, antimicrobial agents may comprise cetyl-trimethylammonium bromide, cetylpyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, especially zinc salts, and acids thereof, heavy metal salts of pyrithione, especially zinc pyrithione, zinc phenolsulfate, farnesol, and combinations thereof. The concentration of the optional deodorant active may range from about 0.001%, from about 0.01%, of from about 0.1%, by weight of the composition to about 20%, to about 10%, to about 5%, or to about 1%, by weight of the composition.

Odor Entrappers

The composition can include an odor entrapper. Suitable odor entrappers for use herein include, for example, solubilized, water-soluble, uncomplexed cyclodextrin. As used herein, the term “cyclodextrin” includes any of the known cyclodextrins such as unsubstituted cyclodextrins containing from six to twelve glucose units, especially, alpha-cyclodextrin, beta-cyclodextrin, gamma-cyclodextrin and/or their derivatives and/or mixtures thereof. The alpha-cyclodextrin consists of six glucose units, the beta-cyclodextrin consists of seven glucose units, and the gamma-cyclodextrin consists of eight glucose units arranged in a donut-shaped ring. The specific coupling and conformation of the glucose units give the cyclodextrins a rigid, conical molecular structure with a hollow interior of a specific volume. The “lining” of the internal cavity is formed by hydrogen atoms and glycosidic bridging oxygen atoms, therefore this surface is fairly hydrophobic. The unique shape and physical-chemical property of the cavity enable the cyclodextrin molecules to absorb (form inclusion complexes with) organic molecules or parts of organic molecules which can fit into the cavity. Many perfume molecules can fit into the cavity.

Cyclodextrin molecules are described in U.S. Pat. Nos. 5,714,137, and 5,942,217. Suitable levels of cyclodextrin are from about 0.1% to about 5%, alternatively from about 0.2% to about 4%, alternatively from about 0.3% to about 3%, alternatively from about 0.4% to about 2%, by weight of the composition.

Buffering Agent

The composition can include a buffering agent which may be alkaline, acidic or neutral. The buffer can be used in the composition for maintaining the desired pH. The composition may have a pH from about 3.25 to about 6, from about 3.5 to about 5.5, or from about 3.7 to about 5.

Suitable buffering agents include, for example, hydrochloric acid, sodium hydroxide, potassium hydroxide, and combinations thereof.

The compositions can contain at least about 0%, alternatively at least about 0.001%, alternatively at least about 0.01%, by weight of the composition, of a buffering agent. The composition may also contain no more than about 1%, alternatively no more than about 0.75%, alternatively no more than about 0.5%, by weight of the composition, of a buffering agent.

The deodorant compositions of the present invention may have a pH of at least about 3.25. In some embodiments, the deodorant may have a pH of at least about 3.5 or at least about 3.7.

Chelator

The deodorant compositions may comprise a chelator. Specific and/or additional chelators in the present invention may include, but are not limited to, diethylenetriaminepentaacetic acid (DTPA), diethylenetriaminepentakis (methylenephosphonic acid) (DTPMP), desferrioxamine, their salts and combinations thereof, EDTA, DPTA, EDDS, enterobactin, desferrioxamine, HBED, and combinations thereof. The amount of chelant, by weight of composition, may be from about to about 4%.

Solubilizer or Emulsifiers

The composition can contain a solubilizer or emulsifier. A suitable solubilizer or emulsifier can be, for example, a surfactant, such as a no-foaming or low-foaming surfactant. Suitable surfactants are nonionic surfactants, cationic surfactants, amphoteric surfactants, zwitterionic surfactants, and mixtures thereof.

Suitable solubilizers and emulsifiers include, for example, hydrogenated castor oil, polyoxyethylene 2 stearyl ether, polyoxyethylene 20 stearyl ether, PEG/PPG-18/18 Dimethicone and combinations thereof. One suitable emuslifier that may be used in the present composition is PEG/PPG-18/18 Dimethicone.

When the solubilizing agent is present, it is typically present at a level of from about 0.01% to about 15%, alternatively from about 0.01% to about 10%, alternatively from about 0.05% to about 5%, alternatively from about 0.01% to about 3%, by weight of the composition.

Preservatives

The composition can include a preservative. The preservative is included in an amount sufficient to prevent spoilage or prevent growth of inadvertently added microorganisms for a specific period of time, but not sufficient enough to contribute to the odor neutralizing performance of the composition. In other words, the preservative is not being used as the antimicrobial compound to kill microorganisms on the surface onto which the composition is deposited in order to eliminate odors produced by microorganisms. Instead, it is being used to prevent spoilage of the composition in order to increase shelf-life.

The preservative can be any organic preservative material which will not cause damage to fabric appearance, e.g., discoloration, coloration, bleaching. Suitable water-soluble preservatives include organic sulfur compounds, halogenated compounds, cyclic organic nitrogen compounds, low molecular weight aldehydes, parabens, propane diaol materials, isothiazolinones, quaternary compounds, benzoates, low molecular weight alcohols, dehydroacetic acid, phenyl and phenoxy compounds, or mixtures thereof.

Non-limiting examples of commercially available water-soluble preservatives include a mixture of about 77% 5-chloro-2-methyl-4-isothiazolin-3-one and about 23% 2-methyl-4-isothiazolin-3-one, a broad spectrum preservative available as a 1.5% aqueous solution under the trade name Kathon® CG by Rohm and Haas Co.; 5-bromo-5-nitro-1,3-dioxane, available under the tradename Bronidox L® from Henkel; 2-bromo-2-nitropropane-1,3-diol, available under the trade name Bronopol® from Inolex; 1,1′-hexamethylene bis(5-(p-chlorophenyl)biguanide), commonly known as chlorhexidine, and its salts, e.g., with acetic and digluconic acids; a 95:5 mixture of 1,3-bis(hydroxymethyl)-5,5-dimethyl-2,4-imidazolidinedione and 3-butyl-2-iodopropynyl carbamate, available under the trade name Glydant Plus® from Lonza; N-[1,3-bis(hydroxymethyl)2,5-dioxo-4-imidazolidinyl]-N,N′-bis(hydroxy-methyl) urea, commonly known as diazolidinyl urea, available under the trade name Germall® II from Sutton Laboratories, Inc.; N,N″-methylenebis{N′-[1-(hydroxymethyl)-2,5-dioxo-4-imidazolidinyl]urea}, commonly known as imidazolidinyl urea, available, e.g., under the trade name Abiol® from 3V-Sigma, Unicide U-13® from Induchem, Germall 115® from Sutton Laboratories, Inc.; polymethoxy bicyclic oxazolidine, available under the trade name Nuosept® C from Hills America; formaldehyde; glutaraldehyde; polyaminopropyl biguanide, available under the trade name Cosmocil CQ® from ICI Americas, Inc., or under the trade name Mikrokill® from Brooks, Inc; dehydroacetic acid; and benzsiothiazolinone available under the trade name Koralone™ B-119 from Rohm and Hass Corporation.

Suitable levels of preservative can range from about 0.0001% to about 0.5%, alternatively from about 0.0002% to about 0.2%, alternatively from about 0.0003% to about 0.1%, by weight of the composition.

Emollient

Emollients of the present invention may include, but are not limited to, certain liquid triglycerides, certain monoalkylglycol dialkyl acid esters and silicones.

As consumers seek more natural ingredients in their deodorants, one approach to formulation is to use emollients derived from natural oils. Emollients derived from natural oils are derived from plant sources, such as palm oil or coconut oil. One example of an emollient derived from natural oils may be a liquid triglyceride, defined as liquid at 25° C. Thus, products that hope to emphasize natural ingredients may have a significant amount of a liquid triglyceride, for example. Derived directly from plant sources, liquid triglycerides are often short chains. Longer chain triglycerides may be used as structurants in deodorant or antiperspirant sticks, but the triglycerides of the present invention are liquid at room temperature (25° C.) and tend to be shorter chains, in some embodiments from C8 to C10. Examples may be caprylic/capric triglyceride (coconut oil fractionated) or triheptanoin.

The present inventive deodorant sticks may comprise at least about 25% of one or more liquid triglyceride, in some embodiments, at least about 30%, at least 35%, at least about 40%, at least about 45%, or at least about 50% liquid triglyceride, by weight of the composition. In some embodiments, the deodorant stick comprises from about 25% to about 60%, by weight of the composition, of one or more liquid triglyceride, from about 25% to about 50%, from about 30% to about 50%, from about 35% to about 60%, from about 35% to about 50%, from about 40% to about 60%, or from about 40% to about 50%, by weight of the composition, of one or more liquid triglyceride. In general, the greater amount of liquid in the formulation, the softer the deodorant stick may be. The more solids in the formulation leads to greater hardness. Because achieving a sufficient softness in a deodorant stick with natural ingredients can be a challenge, it can be beneficial to formulate with higher amounts of liquids such as liquid triglyceride. The level of liquid triglyceride as referred to herein may be the sum total of one or more types of liquid triglyceride in a particular deodorant stick.

In some embodiments, additional emollients may be used, such as plant oils (generally used at less than 10%) including olive oil, coconut oil, sunflower seed oil, jojoba seed oil, avocado oil, canola oil, and corn oil. Additional emollients including mineral oil; shea butter, PPG-14 butyl ether; isopropyl myristate; petrolatum; butyl stearate; cetyl octanoate; butyl myristate; myristyl myristate; C12-15 alkylbenzoate (e.g., Finsolv™); octyldodecanol; isostearyl isostearate; octododecyl benzoate; isostearyl lactate; isostearyl palmitate; isobutyl stearate; dimethicone, and any mixtures thereof.

The deodorant composition can comprise a monoalkylglycol dialkyl acid ester solvent at concentrations ranging from about 0% to about 80%, preferably from about 0% to about 60%, more preferably from about 0% to about 50%, by weight of the composition. In some embodiments, the modoalkylglycol dialkyl acid ester may be neopentyl glycol diheptanoate.

The deodorant composition can comprise a silicone solvent at concentrations ranging from about 0% to about 80%, preferably from about 0% to about 60%, more preferably from about 0% to about 50%, by weight of the composition. The volatile silicone of the solvent may be cyclic or linear.

Appropriate silicones may include volatile silicones, referring to those silicone materials which have measurable vapor pressure under ambient conditions. Nonlimiting examples of suitable volatile silicones are described in Todd et al., “Volatile Silicone Fluids for Cosmetics”, Cosmetics and Toiletries, 91:27-32 (1976), which descriptions are incorporated herein by reference. Preferred volatile silicone materials are those having from about 3 to about 7, preferably from about 4 to about 5, silicon atoms.

Cyclic volatile silicones are preferred for use in the antiperspirant compositions herein, and include those represented by the formula:

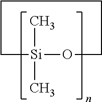

wherein n is from about 3 to about 7, preferably from about 4 to about 5, most preferably 5. These cyclic silicone materials will generally have viscosities of less than about 10 centistokes at 25° C.

Linear volatile silicone materials suitable for use in the antiperspirant compositions include those represented by the formula:

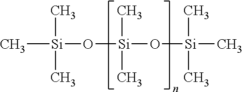

wherein n is from about 1 to about 7, preferably from about 2 to about 3. These linear silicone materials will generally have viscosities of less than about 5 centistokes at 25° C.

Specific examples of volatile silicone solvents suitable for use in the antiperspirant compositions include, but are not limited to, Cyclomethicone D-5 (commercially available from G. E. Silicones), Dow Corning 344, Dow Corning 345 and Dow Corning 200 (commercially available from Dow Corning Corp.), GE 7207 and 7158 (commercially available from General Electric Co.) and SWS-03314 (commercially available from SWS Silicones Corp.).

Structurants

It may be desirable to make the deodorant composition in the form of a solid stick. The solid stick composition may be substantially free of water. The deodorant compositions of the present invention may comprise a suitable concentration of structurants to help provide the compositions with the desired viscosity, rheology, texture and/or product hardness, or to otherwise help suspend any dispersed solids or liquids within the composition.

It is known that to formulate a solid antiperspirant or deodorant stick, the structurants generally have a melting point above 50° C. to provide a stable structure to the stick. The present inventors have discovered that a deodorant stick having at least about 25% of a liquid triglyceride, and that uses a primary structurant that has a melting point of at least about 50° C., in some embodiments from about 50° C. to 70° C. and in still other embodiments from about 50° C. to about while limiting the amount of secondary structurants having a melting point of at least about to 8% or less, can result in a deodorant stick with a hardness from about 80 mm*10 to about 140 mm*10. Such a deodorant stick is able to comprise consumer-perceived natural ingredients, while offering a pleasant consumer experience in terms of its hardness.

The primary structurant in the present invention may have a melting point of at least about in some embodiments from about 50° C. to about 70° C., and in other embodiments from about to about 75° C., and in other embodiments from about 60° C. to 80° C. A primary structurant is defined as the structurant that is present in the composition in the greatest amount (liquid triglycerides are not considered a structurant in this context). Some embodiments may have just a single structurant, so may have only a primary structurant. Other embodiments may have a primary structurant and then secondary structurants, those structurants that are used in a lesser amount than the primary structurant.

The primary structurant may comprise from about 5% to about 20%, in some cases 7-17% of the deodorant stick. The secondary structurants may cumulatively comprise about 12% or less, or about 8% or less of the deodorant stick, in some embodiments less than about 5%, less than about 3%, or less than about 1% of the deodorant stick. In some embodiments, the deodorant stick may be free of or substantially free of any secondary structurants

In some embodiments, some secondary structurants may have a melting point less than 60° C., and then remaining secondary structurants have a melting point of at least about 60° C. The percentage of secondary structurants having a melting point less than 60° C. may not be as significant as the percentage of secondary structurants having a melting point of at least about as the higher melting structurants are what contribute more to the hardness of the deodorant stick. So in some embodiments, the secondary structurants having a melting point of at least about 60° C. may cumulatively comprise 8% or less of the deodorant stick, in some embodiments less than about 5% of the deodorant stick, less than about 3% of the deodorant stick, or less than about 1% of the deodorant stick. In some embodiments, the deodorant stick may be free of or substantially free of any secondary structurants having a melting point of at least about 60° C.

Waxes with melting points between 50° C. and 70° C. include Japan wax, lemon wax, grapefruit wax, beeswax, ceresine, paraffin, hydrogenated jojoba, ethylene glycol distearate, stearyl stearate, palmityl stearate, stearyl behenate, cetearyl behenate, hydrogenated high erucic acid rapeseed oil, and stearyl alcohol.

Waxes with melting points above 70° C. include ozokerite, candelilla, carnauba, espartograss, cork wax, guaruma, rice oil wax, sugar cane wax, ouricury, montan ester wax, sunflower wax, shellac, ozocerite, microcrystalline wax, sasol wax, polyethylenes, polymethylenes, ethylene glycol dipalmitate, ethylene glycol di(12-hydroxystearate), behenyl behenate, glyceryl tribehenate, hydrogenated castor oil (castor wax), and behenyl alcohol.

Waxes with melting points that could vary and possibly fall into either of the two previous groups (depending on factors such as chain length) include C18-C36 triglyceride, Fischer-Tropsch waxes, silicone waxes, C30-50 alkyl beeswax, C20-40 alkyl erucates, C18-38 alkyl hydroxy stearoyl stearates, C20-40 dialkyl esters of dimer acids, C16-40 alkyl stearates, C20-40 alkyl stearates, cetyl ester wax, and spermaceti.

Suitable gelling agents include fatty acid gellants such as fatty acid and hydroxyl or hydroxyl fatty acids, having from about 10 to about 40 carbon atoms, and ester and amides of such gelling agents. Non-limiting examples of such gelling agents include, but are not limited to, 12-hydroxystearic acid, 12-hydroxylauric acid, 16-hydroxyhexadecanoic acid, behenic acid, eurcic acid, stearic acid, caprylic acid, lauric acid, isostearic acid, and combinations thereof. Preferred gelling agents are 12-hydroxystearic acid, esters of 12-hydroxystearic acid, amides of 12-hydroxystearic acid and combinations thereof.

These solid structurants include gelling agents, and polymeric or non-polymeric or inorganic thickening or viscosifying agents. Such materials will typically be solids under ambient conditions and include organic solids, crystalline or other gellants, inorganic particulates such as clays or silicas, or combinations thereof.

The concentration and type of solid structurant selected for use in the deodorant compositions will vary depending upon the desired product hardness, rheology, and/or other related product characteristics. For most structurants suitable for use herein, the total structurant concentration ranges from about 5% to about 35%, more typically from about 10% to about 30%, or from about 7% to about 20%, by weight of the composition.

Non-limiting examples of suitable structurants include stearyl alcohol and other fatty alcohols; hydrogenated castor wax (e.g., Castorwax MP80, Castor Wax, etc.); hydrocarbon waxes include paraffin wax, beeswax, carnauba, candelilla, spermaceti wax, ozokerite, ceresin, baysberry, synthetic waxes such as Fisher-Tropsch waxes, and microcrystalline wax; polyethylenes with molecular weight of 200 to 1000 daltons; solid triglycerides; behenyl alcohol, or combinations thereof. The deodorant stick may further comprise one or more structural elements selected from the group consisting of waxes, natural oils, coconut oil, fractionated coconut oil, jojoba seed oil, olive oil, soybean oil, sunflower oil, and combinations thereof.

Other non-limiting examples of primary structurants suitable for use herein are described in U.S. Pat. No. 5,976,514 (Guskey et al.) and U.S. Pat. No. 5,891,424 (Bretzler et al.), the descriptions of which are incorporated herein by reference.

Non-limiting examples of suitable additional structurants include stearyl alcohol and other fatty alcohols; hydrogenated castor wax (e.g., Castorwax MP80, Castor Wax, etc.); hydrocarbon waxes include paraffin wax, beeswax, carnauba, candelilla, spermaceti wax, ozokerite, ceresin, baysberry, synthetic waxes such as Fisher-Tropsch waxes, and microcrystalline wax; polyethylenes with molecular weight of 200 to 1000 daltons; and solid triglycerides; behenyl alcohol, or combinations thereof.

Other non-limiting examples of additional structurants suitable for use herein are described in U.S. Pat. No. 5,976,514 (Guskey et al.) and U.S. Pat. No. 5,891,424 (Bretzler et al.).

Antimicrobials

The present invention may include one or more antimicrobial compositions. For example, antimicrobials may include, without being limited to, octenidine dihydrochloride (octenidine HCl), hexamidine, polyvinyl formate, salicylic acid, mandelic acid, niacinamide, cinnamon essential oil, cinnamon bark essential oil, cinnamic aldehyde, piroctone olamine, polydialyldimethylammonium chloride, polyquaternium, and combinations thereof. baking soda, hexamidine, thymol, cinnamon essential oil, cinnamon bark essential oil, cinnamic aldehyde, polyvinyl formate, salicylic acid, niacinamide, phenoxyethanol, eugenol, linolenic acid, dimethyl succinate, citral, triethyl citrate, sepiwhite, a substituted or unsubstituted 2-pyridinol-N-oxide material (piroctone olamine), and combinations thereof. The deodorant stick may be free of or substantially free of a substituted or unsubstituted 2-pyridinol-N-oxide material.

In general, the total amount of antimicrobial used in the present invention may be from about to about 30%, by weight, of the deodorant. Some antimicrobials may be used in amounts as low as about 0.0.03%, by weight of the deodorant composition, such as if using octenidine dihydrochloride as the primary antimicrobial, while others could be as high as about 25% of the primary antimicrobial (primary antimicrobial being the antimicrobial present in the composition in the highest amount).

While numerous antimicrobials exhibit efficacy against two main bacteria strains that antiperspirants and deodorants try to address, due to regulatory and safety reasons, there are sometimes limits as to how much of a particular antimicrobial may be put into an antiperspirant or deodorant formula. Therefore, there may be a need for multiple antimicrobials to work together in a formula to deliver enough long-term odor protection.

The deodorant compositions as described herein can contain a structurant, an antimicrobial, a perfume, and additional chassis ingredient(s). The deodorant composition may further comprise other optional ingredient(s). The composition can be in the form of a deodorant cream. The compositions can be in the form of a solid stick. The compositions may be free of dipropylene glycol, added water, castor wax, or any combination thereof. The deodorant composition may be anhydrous. The deodorant composition may be free of added water.

Hardness

The deodorant compositions of the present invention may have a product or stick hardness from about 60 mm*10 to about 160 mm*10, as measured by penetration with ASTM D-1321 needle (see Hardness test method below). In some embodiments, the product hardness may be from about 80 to about 140 mm*10, and in others from about 85 to about 110 mm*10.

Perfume

Perfumes are often a combination of many raw materials, known as perfume raw materials. Any perfume suitable for use in a deodorant composition may be used herein. In some embodiments, the deodorant composition may be free of, or substantially free of a synthetic fragrance. A synthetic fragrance is one mostly derived through chemical synthesis where the starting material is no longer intact, but is converted to the new fragrance chemical.

A natural or essential oil fragrance is a result of natural sources wherein the fragrance material is not altered (chemically modified) but extracted from its natural source. These sources can include, but are not limited to, bark, flowers, blossoms, fruits, leaves, resins, roots, bulbs, and seeds. Natural or essential oils go through an extraction process instead of chemical synthesis. Extraction processes include, but are not limited to, maceration, solvent extraction, distillation, expression of a fruit peel, or effleurage.

Additional Chassis Ingredients

### Starch

The deodorant composition may comprise a starch powder for dry feel or wetness absorption. Examples include but are not limited to arrowroot powder, tapioca starch, and corn starch.

Non-Volatile Organic Fluids

Non-volatile organic fluids may be present, for example, in an amount of about 15% or less, by weight of the composition.

Non-limiting examples of nonvolatile organic fluids include mineral oil, PPG-14 butyl ether, isopropyl myristate, petrolatum, butyl stearate, cetyl octanoate, butyl myristate, myristyl myristate, C12-15 alkylbenzoate (e.g., Finsolv™), octyldodecanol, isostearyl isostearate, octododecyl benzoate, isostearyl lactate, isostearyl palmitate, and isobutyl stearate.

Propellant

The compositions described herein can include a propellant. Some examples of propellants include compressed air, nitrogen, inert gases, carbon dioxide, and mixtures thereof. Propellants may also include gaseous hydrocarbons like propane, n-butane, isobutene, cyclopropane, and mixtures thereof. Halogenated hydrocarbons like 1,1-difluoroethane may also be used as propellants. Some non-limiting examples of propellants include 1,1,1,2,2-pentafluoroethane, 1,1,1,2-tetrafluoroethane, 1,1,1,2,3,3,3-heptafluoropropane, trans-1,3,3,3-tetrafluoroprop-1-ene, dimethyl ether, dichlorodifluoromethane (propellant 12), 1,1-dichloro-1,1,2,2-tetrafluoroethane (propellant 114), 1-chloro-1,1-difluoro-2,2-trifluoroethane (propellant 115), 1-chloro-1,1-difluoroethylene (propellant 142B), 1,1-difluoroethane (propellant 152A), monochlorodifluoromethane, and mixtures thereof. Some other propellants suitable for use include, but are not limited to, A-46 (a mixture of isobutane, butane and propane), A-31 (isobutane), A-17 (n-butane), A-108 (propane), AP70 (a mixture of propane, isobutane and n-butane), AP40 (a mixture of propane, isobutene and n-butane), AP30 (a mixture of propane, isobutane and n-butane), and 152A (1,1 diflouroethane). The propellant may have a concentration from about 15%, 25%, 30%, 32%, 34%, 35%, 36%, 38%, 40%, or 42% to about 70%, 65%, 60%, 54%, 52%, 50%, 48%, 46%, 44%, or 42%, or any combination thereof, by weight of the total fill of materials stored within the container.

Other Optional Ingredients

The deodorant compositions of the present invention may further comprise any optional material that is known for use in antiperspirant and deodorant compositions or other personal care products, or which is otherwise suitable for topical application to human skin.

One example of an optional ingredient is a scent expression material. Scent expression or release technology may be employed with some or all of the fragrance materials to define a desired scent expression prior to use and during use of the deodorant products. Such scent expression or release technology can include cyclodextrin complexing material, like beta cyclodextrin. Other materials, such as, for example, starch or silica-based matrices or microcapsules may be employed to “hold” fragrance materials prior to exposure to bodily-secretions (e.g., perspiration). The encapsulating material may have release mechanisms other than via a solvent; for example, the encapsulating material may be frangible, and as such, rupture or fracture with applied shear and/or normal forces encountered during application and while wearing. A microcapsule may be made from many materials, one example is polyacrylates.

Another example of optional materials are clay mineral powders such as talc, mica, sericite, silica, magnesium silicate, synthetic fluorphlogopite, calcium silicate, aluminum silicate, bentonite and montomorillonite; pearl pigments such as alumina, barium sulfate, calcium secondary phosphate, calcium carbonate, titanium oxide, finely divided titanium oxide, zirconium oxide, zinc oxide, hydroxy apatite, iron oxide, iron titrate, ultramarine blue, Prussian blue, chromium oxide, chromium hydroxide, cobalt oxide, cobalt titanate, titanium oxide coated mica; organic powders such as polyester, polyethylene, polystyrene, methyl methacrylate resin, cellulose, 12-nylon, 6-nylon, styrene-acrylic acid copolymers, poly propylene, vinyl chloride polymer, tetrafluoroethylene polymer, boron nitride, fish scale guanine, laked tar color dyes, laked natural color dyes; and combinations thereof.

Nonlimiting examples of other optional materials include emulsifiers, distributing agents, antimicrobials, pharmaceutical or other topical active, preservatives, surfactants, chelants, and so forth. Examples of such optional materials are described in U.S. Pat. No. 4,049,792 (Elsnau); U.S. Pat. No. 5,019,375 (Tanner et al.); and U.S. Pat. No. 5,429,816 (Hofrichter et al.); which descriptions are incorporated herein by reference.

In some embodiments, the compositions of the present invention may be free of and/or substantially free of aluminum. In some embodiments, the compositions of the present invention may be free of and/or substantially free of citric acid. In some embodiments, the compositions of the present invention may be free and/or substantially free of a starch or starch derivative. In some embodiments, the compositions may be free of and/or substantially free of glycol. In some embodiments, the compositions of the present invention may be free of and/or substantially free of water.

### Examples

### MIC of Various Carboxylic Acids

The following MIC (Minimum Inhibitory Concentration) was done on various carboxylic acids. The MIC is the lowest concentration of active compound at which no growth of the microorganism is observed macroscopically. In this case, the MIC was tested in two different growth media. The results below indicate that some carboxylic acids can have a greater ability to inhibit bacteria growth than others. Furthermore, some carboxylic acids may have a greater ability to inhibit bacteria at a low pH. Azelaic Acid, Capryloyl Salicylic Acid, Mandelic Acid and Hydroxycapric acid below show the greatest potential to inhibit bacteria at an acidic pH.

TABLE 1

| | | MIC | MIC |
|---|---|---|---|
| | Type of Acid | S. epi pH 4.2 | S. epi pH 7.2 |
| Kojic Acid (1% in H₂O) | Pyranone | >500 ppm | >500 ppm |
| Azelaic Acid | Dicarboxylic | 125 ppm | >500 ppm |
| (1% in EtOH) | Acid | | |
| Capryloyl Salicylic Acid | Beta Hydroxy | 4 ppm | 125 ppm |
| (1% in EtOH) | Acid | | |
| Protocatechuic Acid | Dihydroxyl | 500 ppm | >500 ppm |
| (1% in EtOH) | Benzoic Acid | | |
| Mandelic Acid | Alpha Hydroxy | 312 ppm | >2500 ppm |
| (50 mg in H₂O) | Acid | | |
| Hydroxycapric Acid | Alpha Hydroxy | 15.6 ppm | >2500 ppm |
| (40 mg in EtOH) | Acid | | |

Aqueous-Glycol Formulations with Various Carboxylic Acids

Examples 1-14 in Table 2 are inventive and comparative clear gel formulations made with various carboxylic acids. The formulations are water-in-oil emulsions and are made in the following manner. The materials in the aqueous phase are mixed using conventional mixing techniques. After all ingredients have been added, the pH of the aqueous phase is adjusted with HCl or NaOH to a pH between 3.5 and 4.0. The silicone phase is mixed using conventional mixing techniques. To create the emulsion, the water phase is added in a dropwise fashion via a separation funnel to the oil phase with strong agitation of the silicone phase using an overhead mixer with mixing blade. After all of the water phase has been added to the formulation, the emulsion is subsequently milled in a high shear homogenizer to create a solid gel microemulsion.

TABLE 2

| | | | | | | | | | | | | | | |
|---|---|---|---|---|---|---|---|---|---|---|---|---|---|---|
| | Com- | Com- | | Com- | Com- | | Com- | | Com- | Com- | Com- | Com- | | Com- |
| | par- | par- | Inven- | par- | par- | Inven- | par- | Inven- | par- | par- | par- | par- | Inven- | par- |
| | ative | ative | tive | ative | ative | tive | ative | tive | ative | ative | ative | ative | tive | ative |
| Common/Trade | Ex. 1 | Ex. 2 | Ex. 3 | Ex. 4 | Ex. 5 | Ex. 6 | Ex. 7 | Ex. 8 | Ex. 9 | Ex. 10 | Ex. 11 | Ex. 12 | Ex. 13 | Ex. 14 |
| Name | % | % | % | % | % | % | % | % | % | % | % | % | % | % |

Aqueous Phase

| | | | | | | | | | | | | | | |
|---|---|---|---|---|---|---|---|---|---|---|---|---|---|---|
| Dipropylene | 36.55 | 36.55 | 36.55 | 33.65 | 33.65 | 33.65 | 33.65 | 33.65 | 33.65 | 33.65 | 33.65 | 33.65 | 33.65 | 33.65 |
| Glycol | | | | | | | | | | | | | | |
| Propylene | 12.5 | 12.5 | 12.5 | 12.5 | 12.5 | 12.5 | 12.5 | 12.5 | 12.5 | 12.5 | 12.5 | 12.5 | 12.5 | 12.5 |
| Glycol | | | | | | | | | | | | | | |
| Water | Q.S. | Q.S. | Q.S. | Q.S. | Q.S. | Q.S. | Q.S. | Q.S | Q.S. | Q.S. | Q.S. | Q.S. | Q.S. | Q.S. |
| Sodium | 0.75 | 0.75 | 0.75 | 0.75 | 0.75 | 0.75 | 0.75 | 0.75 | 0.75 | 0.75 | 0.75 | 0.75 | 0.75 | 0.75 |
| Chloride | | | | | | | | | | | | | | |
| Lactic Acid | | | | 1.66 | | | | | | | | | | |
| (90%) | | | | | | | | | | | | | | |
| Citric Acid | | | | | 1.5 | | | | | | | | | |
| Mandelic Acid | | | 1.5 | | | 1.5 | | | | | | | | |
| Maltobionic | | | | | | | 1.5 | | | | | | | |
| Acid | | | | | | | | | | | | | | |
| Hydroxycapric | | | | | | | | 1.5 | | | | | | |
| Acid | | | | | | | | | | | | | | |
| Lactobionic | | | | | | | | | 1.5 | | | | | |
| Acid | | | | | | | | | | | | | | |
| Glycolic Acid | | | | | | | | | | 2.14 | | | | |
| (70%) | | | | | | | | | | | | | | |
| Malic Acid | | | | | | | | | | | 1.5 | | | |
| Tartaric Acid | | | | | | | | | | | | 1.5 | | |
| Salicylic Acid | | | | | | | | | | | | | 1.5 | |
| Ursolic Acid | | | | | | | | | | | | | | 1.5 |
| Adjust to pH | Adjust | Adjust | Adjust | Adjust | Adjust | Adjust | Adjust | Adjust | Adjust | Adjust | Adjust | Adjust | Adjust | Adjust |
| 3.5-4.0 with | to | to | to | to | to | to | to | to | to | to | to | to | to | to |
| HCl or NaOH | pH 4 | pH 3.9 | pH 3.7 | pH 3.7 | pH 3.7 | pH 3.7 | pH 3.7 | pH 3.7 | pH 3.7 | pH 3.7 | pH 3.7 | pH 3.7 | pH 3.7 | pH 4 |
| Sensidin DO | | 0.5 | 0.5 | | | | | | | | | | | |
| from Ashland | | | | | | | | | | | | | | |
| containing | | | | | | | | | | | | | | |
| 1.6% | | | | | | | | | | | | | | |
| Octenidine | | | | | | | | | | | | | | |
| dihydrochloride | | | | | | | | | | | | | | |

Silicone Phase

| | | | | | | | | | | | | | | |
|---|---|---|---|---|---|---|---|---|---|---|---|---|---|---|
| DC5225C | 8.0 | | | 8.0 | 8.0 | 8.0 | 8.0 | 8.0 | 8.0 | 8.0 | 8.0 | 8.0 | 8.0 | 8.0 |
| (Cyclo- | | | | | | | | | | | | | | |
| pentasiloxane | | | | | | | | | | | | | | |
| (and) PEG/ | | | | | | | | | | | | | | |
| PPG-18/18 | | | | | | | | | | | | | | |
| Dimethicone) | | | | | | | | | | | | | | |
| Cyclo- | 3.0 | 3.0 | 3.0 | 3.0 | 3.0 | 3.0 | 3.0 | 3.0 | 3.0 | 3.0 | 3.0 | 3.0 | 3.0 | 3.0 |
| pentasiloxane | | | | | | | | | | | | | | |
| Dimethicone | 5.0 | 5.0 | 5.0 | 5.0 | 5.0 | 5.0 | 5.0 | 5.0 | 5.0 | 5.0 | 5.0 | 5.0 | 5.0 | 5.0 |
| 10 cst | | | | | | | | | | | | | | |
| Perfume | 0.3 | 0.3 | 0.3 | 0.3 | 0.3 | 0.3 | 0.3 | 0.3 | 0.3 | 0.3 | 0.3 | 0.3 | 0.3 | 0.3 |

The products shown in examples 1-14 in Table 2 were tested for their resistance to the growth of bacteria by the Finished Product Soleris Method in a 1:10 dilution with artificial eccrine, pH 6, non-stabilized as provided by Pickering Solutions.

Table 3 shows that only certain carboxylic acids are able to produce a strong resistance to the growth of bacteria. Specifically, hydroxycapric acid, mandelic acid, and salicylic acid had a strong ability to prevent the growth of bacteria, as indicated by the higher detection times reported for those particular acids. When indicated, ND refers to a product had increased hostility such that the product inhibited bacteria growth to beyond the time allowance of the test.

TABLE 3

| Sample Description | DT 0 hr. | DT 3 hr. | DT 6 hr. | DT 24 hr. |
|---|---|---|---|---|
| Comparative Example 1 Aluminum Free Clear Gel Control | 6.5 | 6.5 | 6.3 | 5.9 |
| in 1:10 dilution | | | | |
| Comparative Example 4 with 1.5% Lactic Acid in 1:10 | 8.8 | 11.3 | 12.1 | 17.3 |
| dilution | | | | |
| Comparative Example 5 with 1.5% Citric Acid in 1:10 | 7.3 | 7.2 | 8.0 | 12.2 |
| dilution | | | | |
| Inventive Example 6 with 1.5% Mandelic Acid in 1:10 | 7.3 | 9.0 | 10.8 | ND* |
| dilution | | | | |
| Comparative Example 7 with 1.5% Maltobionic Acid in | 7.1 | 7.5 | 8.0 | 14.1 |
| 1:10 dilution | | | | |
| Inventive Example 8 with 1.5% Hydroxycapric Acid in 1:10 | 12.5 | 13.5 | 15.6 | ND* |
| dilution | | | | |
| Comparative Example 9 with 1.5% Lactobionic Acid in 1:10 | 7.4 | 7.2 | 7.9 | 10.6 |
| dilution | | | | |
| Comparative Example 10 with 1.5% Glycolic Acid in 1:10 | 7.3 | 7.7 | 8.2 | 13.8 |
| dilution | | | | |
| Comparative Example 11 with 1.5% Malic Acid in 1:10 | 7.1 | 7.1 | 7.7 | 8.4 |
| dilution | | | | |
| Comparative Example 12 with 1.5% Tartaric Acid in 1:10 | 7.5 | 7.7 | 8.1 | 15.4 |
| dilution | | | | |
| Inventive Example 13 with 1.5% Salicyilc Acid in 1:10 | 8.5 | ND* | ND* | ND* |
| dilution | | | | |
| Comparative Example 14 with 1.5% Ursolic Acid in 1:10 | 7.2 | 6.6 | 6.9 | 6.4 |
| dilution | | | | |
| Artificial Perspiration, pH 6, non-stabilized | 6.5 | 6.3 | 6.3 | 7.2 |

Higher detection time (DT) indicates greater hostility
*ND = No bacteria detection after 48 hours

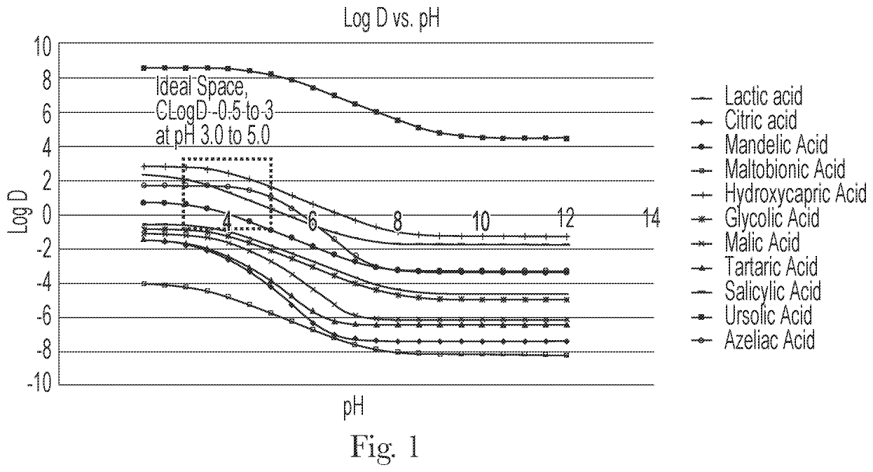

The present inventors have made the surprising discovery that a material's ability to inhibit bacteria relates to the material's lipophilic characteristics. More specifically, the present inventors have discovered that an optimum bacteria-preventing performance occurs with carboxylic acids that have a C log D of −0.5 to 3 at pH 3.0 to 5.0, as shown in FIG. 1. While not being bound by theory, the inventors believe that at pH values from about 3.0-5.0, the carboxylic acids prefer to partition into an octanol phase from water with an equal preference at a value of 0, and 1000:1 preference at a value of 3. This range appears to represent a good range for inhibiting bacterial growth.

The present inventors have identified how to enhance prevention of bacteria when controlling pH with carboxylic acids. Inventive lipophilic carboxylic acids, such as mandelic acid, hydroxycapric acid, azelaic acid, and salicylic acid fall within this identified range and are contained within the box defined in FIG. 1. This space is defined as that of a material having a C Log D in the range of −0.5 to 3 at a pH range of 3.0 to 5.0.

### Combination of Lipophilic Carboxylic Acids and Antimicrobials in Water and Glycol Composition

The present inventors have identified the increased activity of octenidine dihydrochloride when combined with mandelic acid, as shown in Table 4. When indicated, ND refers to a product that had increased hostility such that the product inhibited bacteria growth to beyond the time allowance of the test. As shown, Inventive Example 3 and Inventive Example 6 each had a ND reading at the 24-hour detection time. This was a surprising result, since Comparative Example 2 contained octenidine (Sensidin DO supplied by Ashland) in a formula made in the desired pH range, but, without a carboxylic acid, Comparative Example 2 did not show the same degree of hostility to bacteria. Therefore, the combination of mandelic acid with an octenidine HCl provides a higher degree of hostility. Inventive Example 3 is a preferred formula compared to Inventive Example 6, because the detection time at 3 hour and 6 hour is greater for the formula containing mandelic acid and octenidine (Sensidin DO supplied by Ashland) compared to the formula containing mandelic acid alone.

TABLE 4

| Sample Description | DT 0 hr. | DT 3 hr. | DT 6 hr. | DT 24 hr. |
|---|---|---|---|---|
| Comparative Example 1 Aluminum Free Clear Gel Control in 1:10 | 6.3 | 6.5 | 6.3 | 5.9 |
| dilution | | | | |
| Comparative Example 2 in with 0.5% Sensidin DO 1:10 dilution | 7.3 | 9.1 | 9.7 | 19.7 |
| Inventive Example 3 in with 1.5% Mandelic + 0.5% Sensidin DO 1:10 | 7.3 | 10.8 | 13.8 | ND* |
| dilution | | | | |
| Inventive Example 6 with 1.5% Mandelic Acid in 1:10 dilution | 7.3 | 9.0 | 10.8 | ND* |

Higher detection time (DT) indicates greater hostility.
*ND = No bacteria detection after 48 hours

Examples 15-30 in Table 5 are inventive and comparative clear gel formulations made with various lipophilic carboxylic acids in combination with various antimicrobials. The formulations below are water-in-oil emulsions and are made in the following manner. The materials in the aqueous phase are mixed using conventional mixing techniques. After all ingredients have been added, the pH of the aqueous phase is adjusted with HCl or NaOH to a pH between 3.0 and 4.0. The silicone phase is mixed using conventional mixing techniques. To create the emulsion, the water phase is added in a dropwise fashion via a separation funnel to the oil phase with strong agitation of the silicone phase using an overhead mixer with mixing blade. After all of the water phase has been added to the formulation, the emulsion is subsequently milled in a high shear homogenizer to create a solid gel microemulsion.

TABLE 5

| | | | | | | | | | | | | | | | |
|---|---|---|---|---|---|---|---|---|---|---|---|---|---|---|---|
| | | | | | | | Inven- | Inven- | Inven- | Inven- | Inven- | Inven- | Compar- | Compar- | Compar- |
| Comparative | Inventive | Inventive | Comparative | Comparative | Comparative | Comparative | tive | tive | tive | tive | tive | tive | ative | ative | ative |
| Ex. 15 | Ex. 16 | Ex. 17 | Ex. 18 | Ex. 19 | Ex. 20 | Ex. 21 | Ex. 22 | Ex. 23 | Ex. 24 | Ex. 25 | Ex. 26 | Ex. 27 | Ex. 28 | Ex. 29 | Ex. 30 |

Aqueous Phase

| | | | | | | | | | | | | | | | | |
|---|---|---|---|---|---|---|---|---|---|---|---|---|---|---|---|---|
| Dipropylene | 36.55 | 36.55 | 36.55 | 36.55 | 36.55 | 36.55 | 36.55 | 36.55 | 36.55 | 36.55 | 36.55 | 36.55 | 36.55 | 36.55 | 36.55 | 36.55 |
| Glycol | | | | | | | | | | | | | | | | |
| Propylene Glycol | 12.5 | 12.5 | 12.5 | 12.5 | 12.5 | 12.5 | 12.5 | 12.5 | 12.5 | 12.5 | 12.5 | 12.5 | 12.5 | 12.5 | 12.5 | 12.5 |
| Water | Q.S. | Q.S. | Q.S | Q.S. | Q.S. | Q.S. | Q.S. | Q.S | Q.S. | Q.S. | Q.S. | Q.S. | Q.S. | Q.S. | Q.S. | Q.S. |
| Mandelic Acid | | 1.5 | | | | | | 1.5 | 1.5 | 1.5 | | | | | | |
| Azelaic Acid | | | 1.5 | | | | | | | | 1.5 | 1.5 | 1.5 | | | |
| Succinic Acid | | | | 1.5 | | | | | | | | | | 1.5 | 1.5 | 1.5 |
| Adjust to pH 3.5- | Adjust | Adjust | Adjust | Adjust | Adjust | Adjust | Adjust | Adjust | Adjust | Adjust | Adjust | Adjust | Adjust | Adjust | Adjust | Adjust |
| 4.0 with HCl or | to | to | to | to | to | to | to | to | to | to | to | to | to | to | to | to |
| NaOH | pH 3.8 | pH 3.8 | pH 3.8 | pH 3.8 | pH 3.8 | pH 3.8 | pH 3.8 | pH 3.8 | pH 3.8 | pH 3.8 | pH 3.8 | pH 3.8 | pH 3.8 | pH 3.8 | pH 3.8 | pH 3.8 |
| Sensidin Pure | | | | | 0.15 | | | 0.15 | | | 0.15 | | | 0.15 | | |
| containing 30% | | | | | | | | | | | | | | | | |
| Octenidine | | | | | | | | | | | | | | | | |
| dihydrochloride | | | | | | | | | | | | | | | | |
| in 1,3- | | | | | | | | | | | | | | | | |
| propanediol | | | | | | | | | | | | | | | | |
| Piroctone | | | | | | 0.2 | | | 0.2 | | | 0.2 | | | 0.2 | |
| Olamine | | | | | | | | | | | | | | | | |
| Mirapol 100S | | | | | | | 0.2 | | | 0.2 | | | 0.2 | | | 0.2 |
| from Rhodia | | | | | | | | | | | | | | | | |
| containing 33% | | | | | | | | | | | | | | | | |
| POLYMER OF | | | | | | | | | | | | | | | | |
| DIALLYLDIME | | | | | | | | | | | | | | | | |
| THYLAMMONIUM | | | | | | | | | | | | | | | | |
| CHLORIDE | | | | | | | | | | | | | | | | |
| (DADMAC) in | | | | | | | | | | | | | | | | |
| water | | | | | | | | | | | | | | | | |

Silicone Phase

| | | | | | | | | | | | | | | | | |
|---|---|---|---|---|---|---|---|---|---|---|---|---|---|---|---|---|
| DC5225C | 8.0 | 8.0 | 8.0 | 8.0 | 8.0 | 8.0 | 8.0 | 8.0 | 8.0 | 8.0 | 8.0 | 8.0 | 8.0 | 8.0 | 8.0 | 8.0 |
| (Cyclopentasiloxane | | | | | | | | | | | | | | | | |
| (and) PEG/PPG- | | | | | | | | | | | | | | | | |
| 18/18 Dimethicone) | | | | | | | | | | | | | | | | |
| Cyclopentasiloxane | 3.0 | 3.0 | 3.0 | 3.0 | 3.0 | 3.0 | 3.0 | 3.0 | 3.0 | 3.0 | 3.0 | 3.0 | 3.0 | 3.0 | 3.0 | 3.0 |
| Dimethicone 10 cst | 5.0 | 5.0 | 5.0 | 5.0 | 5.0 | 5.0 | 5.0 | 5.0 | 5.0 | 5.0 | 5.0 | 5.0 | 5.0 | 5.0 | 5.0 | 5.0 |
| Perfume | 0.3 | 0.3 | 0.3 | 0.3 | 0.3 | 0.3 | 0.3 | 0.3 | 0.3 | 0.3 | 0.3 | 0.3 | 0.3 | 0.3 | 0.3 | 0.3 |

The products shown in examples 15-30 in Table 5 were tested for their resistance to the growth of bacteria by the Finished Product Soleris Method in a 1:10 dilution with artificial eccrine, pH 6, non-stabilized as provided by Pickering Solutions.

Table 6 shows that the combination of the lipophilic carboxylic acids mandelic acid and azelaic acid can be desirable when combined with an antimicrobial such as octenidine dihydrochloride or piroctone olamine. The data table shows that the detection time is higher when the materials are combined vs. using either the lipophilic carboxylic acid or antimicrobial alone. In Table 6, comparing Comparative Example 19 (containing Sensidin Pure delivering 0.05% octenidine dihydrochloride but no carboxylic acid) to Inventive Examples 22 and 25, the detection time for the combination has a significant increase. Comparing Comparative Example 19 with Comparative Example 28 (a non-lipophilic carboxylic acid plus Sensidin Pure) shows only a small increase in detection time for the added non-inventive carboxylic acid, showing that carboxylic acids that do not fall within the claimed invention do not have as much increase compared to the use of Sensidin Pure alone.

While previous studies have shown that quaternary antimicrobials will have a higher degree of hostility at an alkaline pH (Wiegand et al, 2015, previously referenced), the present inventors have unexpectedly discovered that some quaternary antimicrobials, such as octenidine dihydrochloride, can provide a synergistic effect and offer a higher degree of hostility at a low pH, such as when combined with lipophilic carboxylic acids of the claimed invention.

TABLE 6

| Sample Description | 0 h | 3 h | 6 h | 24 h |
|---|---|---|---|---|
| Artificial Eccrine, pH 6, non-Stabilized lot 2201079 | 8.9 | 10.0 | 9.1 | 5.7 |
| Comparative Ex. 15 Aluminum Free Clear Gel Control in 1:10 dilution | 8.8 | 9.6 | 9.0 | 7.3 |
| Inventive Ex. 16 containing 1.5% Mandelic Acid in 1:10 dilution | 8.9 | 10.1 | 9.8 | 10.6 |
| Inventive Ex. 17 containing 1.5% Azelaic Acid in 1:10 dilution | 8.9 | 10.1 | 9.8 | 10.2 |
| Comparative Ex. 18 containing 1.5% Succinic Acid in 1:10 dilultion | 9.0 | 9.5 | 9.8 | 9.3 |
| Comparative Ex. 19 containing 0.15% Sensidin Pure in 1:10 dilution | 9.8 | 12.0 | 13.2 | 16.1 |
| Inventive Ex. 22 containing 1.5% mandelic acid + 0.15 Sensidin Pure in 1:10 | 11.8 | 13.9 | 17.3 | ND* |
| dilution | | | | |
| Inventive Ex. 25 containing 1.5% Azelaic Acid + 0.15% Sensidin Pure in 1:10 | 12.3 | 14.2 | 15.8 | ND* |
| dilution | | | | |
| Comparative Ex. 28 containing 1.5% Succinic Acid + 0.15% Sensidin Pure in 1:10 | 11.0 | 13.7 | 13.6 | 20.0 |
| dilution | | | | |

Higher detection time (DT) indicates greater hostility.
*ND = No bacteria detection after 48 hours

In table 7, comparing Comparative Example 20 (containing piroctone olamine alone) to Inventive Examples 23 and 26 (Examples containing lipophilic carboxylic acids and piroctone olamine), the Inventive Examples have longer detection times for most data points. Comparative Example 29 (containing a non-lipophilic carboxylic acid and piroctone olamine) shows no significant increase and even sometimes a decrease in detection time vs. Comparative Example 20 (piroctone olamine alone).

TABLE 7

| Sample Description | 0 h | 3 h | 6 h | 24 h |
|---|---|---|---|---|
| Artificial Eccrine, pH 6, non-Stabilized lot 2201079 | 8.9 | 10.0 | 9.1 | 5.7 |
| Comparative Ex. 15 Aluminum Free Clear Gel Control in 1:10 dilution | 8.8 | 9.6 | 9.0 | 7.3 |
| Inventive Ex. 16 containing 1.5% Mandelic Acid in 1:10 dilution | 8.9 | 10.1 | 9.8 | 10.6 |
| Inventive Ex. 17 containing 1.5% Azelaic Acid in 1:10 dilution | 8.9 | 10.1 | 9.8 | 10.2 |
| Comparative Ex. 18 containing 1.5% Succinic Acid in 1:10 dilultion | 9.0 | 9.5 | 9.8 | 9.3 |
| Comparative Ex. 20 containing 0.2% Piroctone Olamine in 1:10 dilution | 9.1 | 9.4 | 10.6 | 11.4 |
| Inventive Ex. 23 containing 1.5% mandelic acid + 0.2% Piroctone Olamine in 1:10 | 9.6 | 10.0 | 9.8 | 11.8 |
| dilution | | | | |
| Inventive Ex. 26 containing 1.5% Azelaic Acid + 0.2% Piroctone Olamine in 1:10 | 9.8 | 10.8 | 10.0 | 11.3 |
| dilution | | | | |
| Comparative Ex. 29 containing 1.5% Succinic Acid + 0.2% Piroctone Olamine in | 9.7 | 9.4 | 9.9 | 10.8 |
| 1:10 dilution | | | | |

Higher detection time (DT) indicates greater hostility.
*ND = No bacteria detection after 48 hours

### Combination of Lipophilic Carboxylic Acid and Antimicrobial in Composition Free of Water

Inventive and Comparative Examples 31-34 in Table 8 are aluminum free formulations made with mandelic acid in combination with piroctone olamine. The formulations below are anhydrous and are made in the following manner. All materials, excluding powders and perfume are added in any order, mixed using conventional mixing techniques and heated to 85° C. until all the waxes have melted. The remaining powder are added to the formulation and the temperature is reduced to 74° C. The perfume is added to the batch. The formulation is cooled to 58° C. at which point it is poured into canisters suitable for a deodorant product.

TABLE 8

| | | | | | Compar- | Inven- | Inven- | Inven- | Compar- | Inven- | Inven- |
|---|---|---|---|---|---|---|---|---|---|---|---|
| | Compar- | Compar- | Compar- | Inven- | ative | tive | tive | tive | ative | tive | tive |
| | ative | ative | ative | tive | Example | Example | Example | Example | Example | Example | Example |
| | Ex. 31 | Ex. 32 | Ex. 33 | Ex. 34 | 35 | 36 | 37 | 38 | 39 | 40 | 41 |
| Common/Trade | Target | Target | Target | Target | Target | Target | Target | Target | Target | Target | Target |
| name | (%) | (%) | (%) | (%) | (%) | (%) | (%) | (%) | (%) | (%) | (%) |
| Cyclomethicone, | 40.55 | 40.35 | 38.55 | 38.35 | 42.2 | 40.2 | 40.2 | 40.2 | 41.95 | 39.95 | 39.95 |
| DC245, SF1202 | | | | | | | | | | | |
| Fluid AP, Low | 8.0 | 8.0 | 8.0 | 8.0 | 8.0 | 8.0 | 8.0 | 8.0 | 8.0 | 8.0 | 8.0 |
| Odor | | | | | | | | | | | |
| Mineral Oil | 8.0 | 8.0 | 8.0 | 8.0 | 8.0 | 8.0 | 8.0 | 8.0 | 8.0 | 8.0 | 8.0 |
| Petrolatum | 3.0 | 3.0 | 3.0 | 3.0 | 3.0 | 3.0 | 3.0 | 3.0 | 3.0 | 3.0 | 3.0 |
| CO-1897 Stearyl | 17.6 | 17.6 | 17.6 | 17.6 | 17.6 | 17.6 | 17.6 | 17.6 | 17.6 | 17.6 | 17.6 |
| Alcohol NF | | | | | | | | | | | |
| Hydrogenated | 5.0 | 5.0 | 5.0 | 5.0 | 5.0 | 5.0 | 5.0 | 5.0 | 5.0 | 5.0 | 5.0 |
| Castor Oil MP80 | | | | | | | | | | | |
| Deodorized | | | | | | | | | | | |
| Behenyl Alcohol | 0.2 | 0.2 | 0.2 | 0.2 | 0.2 | 0.2 | 0.2 | 0.2 | 0.2 | 0.2 | 0.2 |
| Partially | | 12.0 | | | | | | | | | |
| Carbonated | | | | | | | | | | | |
| Magnesium | | | | | | | | | | | |
| Hydroxide | | | | | | | | | | | |
| Tapioca Starch | 12.0 | | 12.0 | 12.0 | 12.0 | 12.0 | 12.0 | 12.0 | 12.0 | 12.0 | 12.0 |
| Mandelic Acid | | | 2.0 | 2.0 | | 2.0 | | | | | |
| Azelaic Acid | | | | | | | 2.0 | | | 2.0 | |
| Hydroxycapric | | | | | | | | 2.0 | | | 2.0 |
| Acid | | | | | | | | | | | |
| Piroctone Olamine | | 0.2 | | 0.2 | | | | | | | |
| Sensidin Pure | | | | | | | | | 0.25 | 0.25 | 0.25 |
| containing 30% | | | | | | | | | | | |
| Octenidine | | | | | | | | | | | |
| dihydrochloride in | | | | | | | | | | | |
| 1,3-propanediol | | | | | | | | | | | |
| Fragrance and | 2.0 | 2.0 | 2.0 | 2.0 | 2.0 | 2.0 | 2.0 | 2.0 | 2.0 | 2.0 | 2.0 |
| Starch Encapsulate | | | | | | | | | | | |
| beta-cyclodextrin | 2.0 | 2.0 | 2.0 | 2.0 | 2.0 | 2.0 | 2.0 | 2.0 | 2.0 | 2.0 | 2.0 |
| Perfume | 1.65 | 1.65 | 1.65 | 1.65 | | | | | | | |

The products shown in examples 31-34 in table 8 were tested for their resistance to the growth of bacteria by the Finished Product Soleris Method in a 1:10 dilution with artificial eccrine, pH 6, non-stabilized as provided by Pickering Solutions.

Table 9 shows that the combination of the carboxylic acid, such as mandelic acid, can be desirable when combined with an antimicrobial such as piroctone olamine. The data table shows that the detection time is higher for Inventive Example 34 when the materials are combined vs. using mandelic acid alone, as shown in Comparative Example 33. When indicated, ND refers to a product having increased hostility such that the product inhibited bacteria growth to beyond the time allowance of the test.

TABLE 9

| Sample Description | DT 3 hr. | DT 3 hr. | DT 6 hr. | DT 24 hr. |
|---|---|---|---|---|
| Comparative Example 31 free of mandelic acid and free of piroctone | 8.0 | 8.4 | 8.2 | 10.8 |
| olamine in 1:10 dilution | | | | |
| Comparative Example 32 with 0.2% Piroctone Olamine in 1:10 | 8.7 | 15.0 | 30.0 | ND* |
| dilution | | | | |
| Comparative Example 33 with 2% Mandelic Acid in 1:10 dilution | 9.3 | 37.3 | 44.1 | ND* |
| Inventive Example 34 with 2% Mandelic Acid and 0.2% Piroctone | 10.0 | 44.2 | ND* | ND* |
| Olamine in 1:10 dilution | | | | |
| Artificial Eccrine, non-stabilized, pH 6 | 8.9 | 7.8 | 7.4 | 7.2 |

Higher detection time (DT) indicates greater hostility.
*ND = No bacteria detection after 48 hours

Table 10 shows that compositions containing alpha hydroxy acids, including inventive example 36 containing mandelic acid and inventive example 38 containing hydroxycapric acid, have increased hostility compared to example 25 absent of any carboxylic acid. Compositions containing dicarboxylic acid, such as inventive example 37 containing azelaic acid, provide increased hostility compared to comparative example 35 absent of any carboxylic acid or antimicrobial. Additional hostility is seen when lipophilic carboxylic acids are combined with the antimicrobial Sensidin Pure, delivering 0.075% octenidine dihydrochloride. Inventive examples 40 and 41 are significantly higher compared to comparative example with Sensidin Pure absent of the lipophilic carboxylic acid, as seen in the 0 hr., 3 hr. and 6 hr. time point. When indicated, ND refers to a product having increased hostility such that the product inhibited bacteria growth to beyond the time allowance of the test.

TABLE 10

| Sample Description | DT 0 hr. | DT 3 hr. | DT 6 hr. | DT 24 hr. |
|---|---|---|---|---|
| Artificial Eccrine, non-stabilized, pH 6 | 6.9 | 6.5 | 6.4 | 5.2 |
| Comparative Example 35 free of carboxylic acid and free of | 6.8 | 6.7 | 6.4 | 5.3 |
| antimicrobial in 1:100 dilution | | | | |
| Inventive Example 36 with 2% Mandelic Acid in 1:100 dilution | 6.8 | 7.4 | 7.1 | 6.6 |
| Inventive Example 37 with 2% Azelaic Acid in 1:100 dilution | 6.8 | 7.1 | 7.5 | 8.2 |
| Inventive Example 38 with 2% Hydroxycapric Acid in 1:100 | 7.0 | 7.6 | 7.9 | 9.2 |
| dilution | | | | |
| Comparative Example 39 with 0.25% Sensidin Pure in 1:100 | 9.9 | 15.9 | 21.2 | ND* |
| dilution | | | | |
| Inventive Example 40 with 0.25% Sensidin Pure and 2% | 12.3 | 24.5 | 36.3 | ND* |
| Mandelic Acid in 1:100 dilution | | | | |
| Inventive Example 41 with 0.25% Sensidin Pure with 2% | 11.7 | 20.0 | 30.2 | ND* |
| Azelaic Acid in 1:100 dilution | | | | |

Higher detection time (DT) indicates greater hostility.
*ND = No bacteria detection after 48 hours

### In-Vivo Bacteria Detection Via Underarm Swabs

Comparative Example 42 in Table 11 is an anhydrous invisible solid formulation comprising aluminum zirconium antiperspirant active. The materials in the aqueous phase are mixed using conventional mixing techniques. Ingredients are added in the order indicated. After the addition of behenyl alcohol, the mixture is heated to 85° C. until all of the waxes have dissolved. The remaining ingredients are added to the batch and the temperature is lowered to 58° C. at which point the formulation is poured into a suitable deodorant canister and allowed to cool.

TABLE 11

| | Ex. 42 |
|---|---|
| Common/Trade name | Target (%) |
| Cyclomethicone, DC245, SF1202 | 37.05 |
| Dimethicone 50 cst | 5.0 |
| Fluid AP, Low Odor | 2.0 |
| Mineral Oil | 8.0 |
| Petrolatum | 3.0 |
| CO-1897 Stearyl Alcohol NF | 13.75 |
| Hydrogenated Castor Oil MP80 Deodorized | 2.5 |
| Behenyl Alcohol | 0.2 |
| Aluminum Zirconium Trichlorohydrex Gly | 24.0 |
| Talc | 4.0 |
| beta-cyclodextrin | 0.5 |

Inventive Example 43 in Table 12 is a clear gel formulation made with mandelic acid in combination with an Sensidin Pure supplied by Ashland. The formulations below are water-in-oil emulsions and are made in the following manner. The materials in the aqueous phase are mixed using conventional mixing techniques. After all the mandelic acid was added, the pH of the aqueous phase is adjusted with NaOH to a pH of 3.25 followed by the addition of Sensidin Pure. The silicone phase is mixed using conventional mixing techniques. To create the emulsion, the water phase is added in a dropwise fashion via a separation funnel to the oil phase with strong agitation of the silicone phase using an overhead mixer with mixing blade. After all of the water phase has been added to the formulation, the emulsion is subsequently milled in a high shear homogenizer to create a solid gel microemulsion.

TABLE 12

Inventive Ex. 43

Aqueous Phase

| | |
|---|---|
| Dipropylene Glycol | 27.25 |
| Propylene Glycol | 12.5 |
| Water | Q.S. |
| Sodium Chloride | 0.75 |
| Mandelic Acid | 5.5 |
| | Adjust to pH 3.25 with NaOH |
| Sensidin Pure from Ashland containing 30% | 1 |
| Octenidine dihydrochloride in 1,3-propanediol | |

Silicone Phase

| | |
|---|---|
| Piroctone Olamine | |
| DC5225C (Cyclopentasiloxane (and) | 8.0 |
| PEG/PPG-18/18 Dimethicone) | |
| Cyclopentasiloxane | 3.0 |
| Dimethicone 10 cst | 5.0 |

The following data is in-vivo confirmation of the in-vitro Finished product Soleris method, showing that example 43 comprising 5.5% Mandelic Acid and 0.3% Octenidine dihydrochloride can exceed the ability of an aluminum containing invisible solid (Comparative Example 42) and competitive aluminum free Crème comprising 5% mandelic acid, but lacking glycol and an antimicrobial.

Tests subjects recruited for the study were asked to replace their normal underarm product with an aluminum free product for the week prior to the study. The three days prior to the product application, test subjects were instructed to discontinue use of any underarm product, using only the bar soap provided and no scrubbing of the underarms was allowed. Test product was applied at a dosage of 0.3 g per underarm and product assessment occurred over a 5-day period. Bacteria is collected via the Soleris Swab Collection procedure on Day 0 (baseline), 24 hours after Pt use and 24 hours after 4^{th} use and subsequently analyzed for bacteria count via Soleris. Soleris analysis provides Detection Time (DT) which is inversely proportional to living microbial biomass in mixture

Table 13 shows both the data from the in-vitro Finished Product Soleris Method and in-vivo underarm bacteria swabbing shows that the combination of a lipophilic carboxylic acid with an antimicrobial provides superior prevention of underarm bacteria.

TABLE 13

| | | Aluminum Free Clear Gel Comprising | |
|---|---|---|---|
| | | 5.5% Mandelic Acid and 0.3% | Competitive Aluminum Free Crème |
| | Invisible Solid Formulation | Octenidine dihydrochloride | containing 5% Mandelic Acid |
| | Comparative Ex. 42 | Inventive Ex. 43 | Comparative Example 44 |
| Finished Product Soleris Detection | 22.9 | 29.0 | 12.7 |
| Time (DT) at T = 0 | | | |
| 1:10 dilution in Artificial Eccrine, | | | |
| pH 6 | | | |
| Baseline-in-vivo underarm | 7.81 | 8.32 | 8.78 |
| bacterial detection time (Soleris) | | | |
| 24 hr. After 1^{st} Use-in-vivo | 12.24 | 13.63 A | 13.11 |
| underarm bacteria detection time | | | |
| (Soleris) | | | |
| % Increase in Detection Time vs. | 56% | 64% | 49% |
| Baseline after 1^{st} Use | | | |
| 24 hr. After 4^{th} Use-in-vivo | 14.47 C | 15.13 C | 12.99 |
| underarm bacteria detection time | | | |
| (Soleris) | | | |
| % Increase in Detection time vs. | 85% | 82% | 48% |
| Baseline after 4^{th} Use | | | |

Higher detection time (DT) indicates greater hostility.

### Combination of Lipophilic Carboxylic Acids and Antimicrobials in Water and Polyether Composition

Examples 45-47 in Table 14 are inventive and comparative clear gel formulations made with mandelic acid in combination with Sensidin Pure supplied by Ashland and Sensidin Pure without a lipophilic carboxylic acid. The formulations below are polar-in-nonpolar emulsions and are made in the following manner. The materials in the polar phase are mixed using conventional mixing techniques. The nonpolar phase is mixed using conventional mixing techniques. To create the emulsion, the polar phase is added in a dropwise fashion via a separation funnel to the nonpolar phase with strong agitation of the nonpolar phase using an overhead mixer with mixing blade. After all of the polar phase has been added to the formulation, the emulsion is subsequently milled in a high shear homogenizer to create a solid gel microemulsion.

TABLE 14

| | Inventive | Inventive | Comparative |
|---|---|---|---|
| | Ex. 45 | Ex. 46 | Ex. 47 |
| Polar Phase | | | |
| Mandelic Acid | 4 | 2 | |
| Sensidin Pure from | 0.1 | 0.2 | 0.3 |
| Ashland containing | | | |
| 30% Octenidine | | | |
| dihydrochloride in 1,3- | | | |
| propanediol | | | |
| PEG-8 (PEG 400) | | 36 | |
| PEG-12 (PEG 600) | 30 | | 25 |
| Glycerin | 10 | | 15 |
| Sodium Chloride | 0.5 | 0.5 | 0.5 |
| Water | 37.4 | 43.8 | 42.2 |
| Trisodium Citrate | 1 | 0.5 | |
| Dihydrate to adjust pH | | | |
| Polar Phase pH | 3.90 | 3.94 | Not |
| | | | measured |
| Nonpolar Phase | | | |
| Cyclopentasiloxane | 3.25 | 3.25 | 3.25 |
| Dimethicone 10 cst | 5 | 5 | 5 |
| DC5225C | 8 | 8 | 8 |
| (Cyclopentasiloxane | | | |
| (and) PEG/PPG-18/18 | | | |
| Dimethicone) | | | |
| Fragrance | 0.75 | 0.75 | 0.75 |

Odor protection performance of inventive examples 45-46 and comparative example 47 was evaluated by a panel of 5-10 experts who used the product at home. Odor protection performance of each example was tested by comparing to a market aluminum-free deodorant benchmark containing dipropylene glycol, sodium stearate and fragrance among other ingredients. Panelists used the example and the market benchmark for a usage period of 5-7 days. The example was used in one underarm and the market benchmark was used in the other underarm for the entire usage period. At the end of the usage period, panelists were asked to select which product they preferred for protecting them from odor based on their experiences over the usage period. Panelists could also select no preference between the two products for protecting them from odor. Table 15 below shows that inventive examples 45-46 delivered better odor protection than the market benchmark and comparative example 47 delivered poorer odor protection than the market benchmark. For inventive examples 45-46, more panelists preferred the Example for protecting them from odor. For comparative example 47, more panelists preferred the Market Benchmark for protecting them from odor.

Table 15 shows that the combination of a lipophilic carboxylic acid with an antimicrobial provides superior odor protection compared to an antimicrobial without a lipophilic carboxylic acid.

TABLE 15

| | # panelists who | # panelists who | |
|---|---|---|---|
| | preferred | preferred Market | |
| | Example for | Benchmark for | |
| | protecting them | protecting them | # panelists with |
| | from odor | from odor | no preference |
| Inventive | 3 | 0 | 4 |
| Ex. 45 | | | |
| Inventive | 2 | 0 | 4 |
| Ex. 46 | | | |
| Comparative | 1 | 4 | 4 |
| Ex. 47 | | | |

### Test Methods

### Minimum Inhibitory Concentration (MIC)

The MIC or minimum inhibitory concentration test determines antimicrobial activity of a material against a specific bacteria.

The most commonly employed methods are the tube dilution method and agar dilution method. Test products that are not clear or precipitate the growth media are tested by agar dilution method which is similar to the tube dilution method except dilutions are plated on agar.

The tube dilution test is the standard method for determining levels of microbial resistance to an antimicrobial agent. Serial dilutions of the test agent are made in a liquid microbial growth medium which is inoculated with a standardized number of organisms and incubated for a prescribed time. The lowest concentration (highest dilution) of test agent preventing appearance of turbidity (growth) is considered to be the minimal/minimum inhibitory concentration (MIC). At this dilution the test agent is bacteriostatic.

### Finished Product Soleris Method

Sample preparation is done in the following manner. The product to be tested is weighed into a vial at a ratio of 1 part product to 10 parts Artificial Eccrine, custom pH 6, non-stabilized as obtained by Pickering Laboratories (cat. No. 1700-0023). In some cases where products have a higher hostility, the dilution in a particular study may be 1 part deodorant product to 100 parts Artificial Eccrine, custom pH 6, non-stabilized. Products are heated to 72° C. for 30 minutes to melt solid products and facilitate dispersion into the artificial eccrine. Vial is agitated vigorously to disperse the solids, using a Vortex Genie Agitator or comparable equipment. There may be some insoluble media present after dispersion. The dilutions remainder of the procedure is described below in “Analyzing via Soleris”.

### SOLERIS Swab Collection (3 Total Per Underarm):

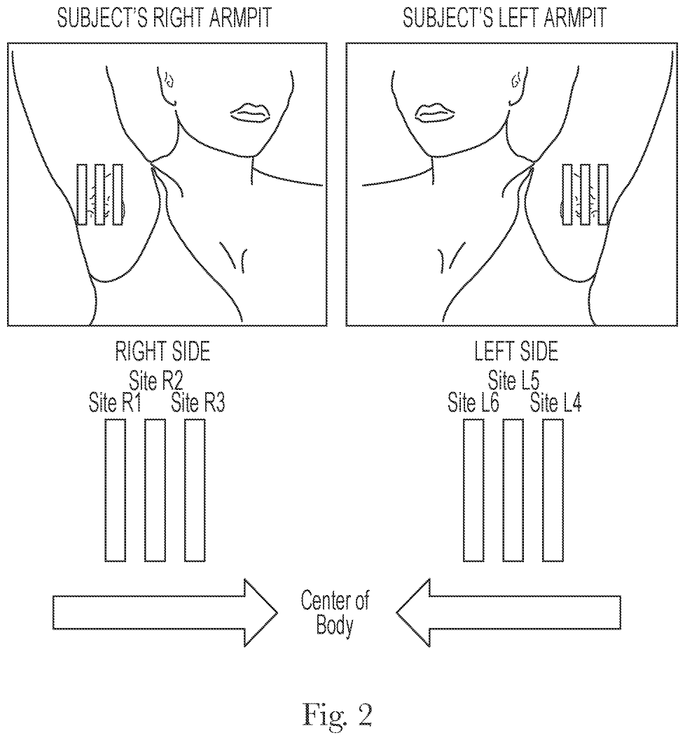

The sampling method is described as follows and references FIG. 2:
1. The subject's armpit will need to be accessible.
2. Practice holding the swab to identify a way that will be comfortable for you. DO NOT touch the swab tip after removal from properly labeled sterile transport tube.
3. Please note: for swabbing you will work from the outside towards the inside body for each armpit. For each subject, you will swab the RIGHT armpit, then the LEFT armpit, according to FIG. 2.
4. The technician will put on a new pair of gloves.
5. Samples will be collected from the RIGHT armpit.
6. The Copan sterile swab is dipped in distilled water.
7. Identify the midline of the Subject's RIGHT armpit. Starting just left of the midline (Site R1) of the Subject's RIGHT armpit, press the swab firmly against the skin and in a vertical direction run the swab up and down for 10 strokes traveling approximately 4 inches per stroke. One stroke is 1 upward motion and 1 downward motion. After each stroke, rotate the swab approximately one-half turn.
8. Once swabbing in Site R1 is complete, the swab will be returned to the labeled sterile transport tube and placed immediately on wet ice.
9. A second Copan swab will be removed from labeled transport tube and dipped in distilled water.
10. Identify the midline (Site R2) of the Subject's RIGHT armpit. Press the swab firmly against the skin and in a vertical direction run the swab up and down the skin for 10 strokes. 1 stroke is (one upward motion and 1 downward motion). After each stroke rotate the swab approximately one-half turn.
11. Once swabbing in Site R2 is complete, the swab will be returned to the labeled sterile transport tube and placed immediately on wet ice.
12. A third Copan swab will be removed from labeled transport tube and dipped in distilled water.
13. Identify the midline of the Subject's RIGHT armpit. Starting just right of the midline (Site R3) of the Subject's RIGHT armpit, press the swab firmly against the skin and in a vertical direction run the swab up and down the skin for 10 strokes. 1 stroke is (one upward motion and 1 downward motion). After each stroke rotate the swab approximately one-half turn.
14. Once swabbing in Site R3 is complete, the swab will be returned to the labeled sterile transport tube and placed immediately on wet ice.
15. Samples will be collected from the LEFT armpit.
16. The Copan sterile swab is dipped in distilled water.
17. Identify the midline of the Subject's LEFT armpit. Starting just right of the midline (Site L4) of the Subject's RIGHT armpit, press the swab firmly against the skin and in a vertical direction run the swab up and down for 10 strokes. One stroke is 1 upward motion and 1 downward motion. After each stroke, rotate the swab approximately one-half turn.
18. Once swabbing in Site L4 is complete, the swab will be placed in the designated swab tube and the tube will be placed immediately on wet ice.
19. A second Copan swab will be dipped in distilled water.
20. Identify the midline (Site L5) of the Subject's LEFT armpit. Press the swab firmly against the skin and in a vertical direction run the swab up and down the skin for 10 strokes. 1 stroke is (one upward motion and 1 downward motion). After each stroke rotate the swab approximately one-half turn.
21. Once swabbing in Site L5 is complete, the swab will be placed in the swab tube and the tube will be placed immediately on wet ice.
22. A third Copan swab will be dipped in distilled water.
23. Identify the midline of the Subject's LEFT armpit. Starting just left of the midline (Site L6) of the Subject's RIGHT armpit, press the swab firmly against the skin and in a vertical direction run the swab up and down the skin for 10 strokes. 1 stroke is (one upward motion and 1 downward motion). After each stroke rotate the swab approximately one-half turn.
24. Once swabbing in Site L6 is complete, the swab will be placed in the swab tube and the tube will be placed immediately on wet ice.
25. Once the subject swabbing is complete, all swabs in their tubes will be placed in a Ziploc bag labeled with the Subject Number and SOLERIS and refrigerated.
26. The technician will change gloves in between subjects.

### Analyzing Via Soleris

S. epidermidis grown overnight on tryptic soy agar (37 C)
Colony from agar is suspended in 25 ml tryptic soy broth in baffled flask, and incubated overnight on shaking platform (37 C)
1 ml of overnight culture is diluted into 9 ml of sterile H₂O (this serves as the inoculum for experimental formulas)
For testing liquid products, 5 ml of experimental formula transferred to 15 ml conical tube. For testing solid formulations, a 48 or 96-well tissue culture plates is used, with each well containing 100 ul of solidified formulations. Alternately, for solid formulations, collect of product and apply 10-20 g of water and let sit at least 1 hour. Moderate agitation may be used to release actives from sample.
50 ul of inoculum (˜10⁶ bacterial cfus) added to 5 ml of formula in conical tube (or 1-5 ul of inoculum in culture plates), and the liquid mixture is incubated on rocking platform or stationary in case of culture plates at room temperature
At 0, 3, 6 and 24 h, 100 ul of mixture is transferred to separate Soleris NF-TVC vial, mixed by inversion and placed into Soleris hardware for continuous 48 h analysis at 34 C per vendors instructions. Alternatively, the mixture can be subjected traditional serial dilution, plating and incubation to determine surviving cfu/ml.
Soleris analysis provides Detection Time (DT) which is inversely proportional to living microbial biomass in mixture
Extension of DT vs base formula control reflects relative microbial hostility of experimental formula and reduction of living microbial biomass

### Hardness Test Method—Penetration Measurement for Deodorant Finished Products

The penetration test is a physical test method that provides a measure of the firmness of waxy solids and extremely thick creams and pastes with penetration values not greater than 250 when using a needle for D1321. The method is based on the American Society for Testing and Materials Methods D-5, D1321 and D217 and DIN 51 579 and is suitable for all solid antiperspirant and deodorant products.

A needle or polished cone of precisely specified dimensions and weight is mounted on the bottom of a vertical rod in the test apparatus. The sample is prepared as specified in the method and positioned under the rod. The apparatus is adjusted so that the point of the needle or cone is just touching the top surface of the sample. Consistent positioning of the rod is critical to the measured penetration value. The rod is then released and allowed to travel downward, driven only by the weight of the needle (or cone) and the rod. Penetration is the tenths of a millimeter travelled following release.

### Apparatus Suggested Type (or Equivalent)

Penetrometer with Timer

Penetrometer Suitable For ASTM D-5 and D-1321 methods; Examples: Precision or Humboldt Universal Penetrometer (Humboldt Manufacturing, Schiller Park, Ill. USA) or Penetrometer Model PNR10 or PNR12 (Petrolab USA or PetroTest GmbH).

### Penetration Needles ANTIPERSPIRANT or DEODORANT SOLIDS can use:

Needles as specified for ASTM Method D-5, NIST Certified, Fisher Scientific #01-512.
Needles as specified for ASTM Method D 1321/DIN 51 579, Officially certified, Taper-Tipped needle, No. H-1310, Humboldt Mfg.

General Instructions—All Penetrometers—Keep the instrument and needles/probes clean at all times, free from dust and grime. When not in use, store needles in a suitable container to avoid damage. Periodic calibration should confirm:

Electronic Timer is correctly set. Verify against an independent stopwatch if unsure.

Shaft falls without visible signs of frictional resistance.

Ensure the total weight of the shaft and needle is 50±0.2 grams when the shaft is in free fall. Note: for modern, automated or digital systems this may be performed automatically and confirmed through annual calibration.

At time of use confirm:

Electronic Timer is correctly set to 5.0 seconds.

The appropriate needle is installed and is clean, straight and without obvious defects (visual inspection)

The penetrometer is level and the shaft is clean, straight and falls freely (visual inspection)

Once level, avoid shifting the position of the unit to maintain level.

### Sample Preparation and Measurement

1. On a deodorant stick that has cooled ambiently to a temperature between 22° C. and 26° C. for at least 24 hours, slice off top ½ inch of product to achieve a flat surface with a wire cutter drawn across the upper lip of the canister.

2. For the first sample to be tested, lubricate the needle by gently wiping with a lint-free tissue coated with a small amount of the product to be tested. This small amount is typically taken from the shaved top.

3. Place the canister in the appropriate location for the measurement. Locate the sample so the needle will penetrate the product 9-11 mm from the inside of the canister wall on the long axis.

4. Using the coarse and fine adjustments, align the height of the penetrometer mechanism head so that the point of the penetrating needle is just touching the surface of the sample.

A weak light at the side of the penetrometer which casts a shadow of the needle on the surface of the sample may be helpful in determining this contact. When a light area on the sample cannot be seen at the end of the tip of the needle's shadow, the needle height over the sample is correctly adjusted. The light should not be strong enough to heat or melt the sample surface. The needle should be just close enough to scratch the sample surface.

5. Perform the penetration measurement at this location by releasing the needle. Record the result.

6. Repeat Steps 2 through 4 at the other test point, i.e., at the other point 9-11 mm inside of the canister wall on the long axis.

To report results, units for penetration are tenths of a millimeter (1/10 mm=100 microns). For example, a result of 80 units is 80 mm*10 or 8 mm. Report the average results of at least 4 total measurements from 2 different sticks, report to the nearest tenth of a millimeter.

### Refractive Index—Measurement of Refractive Index (RI)

A refractometer is the instrument used to measure refractive index (RI). A refractometer measures the extent to which light is bent when it moves from air into a sample and is typically used to determine the refractive index of a liquid sample. Refractive index can be measured by any method suitable to provide an accurate refractive index, with a minimum resolution of 0.0001 nD. There are four main types of refractometers, traditional handheld refractometers, digital handheld refractometers, laboratory or Abbe refractometer and inline process refractometers. A digital handheld refractometer (e.g. AR200 Digital Handheld Refractometer, Reichert) can be used, whereas the instrument is calibrated with a standard solution (typically water). A few drops of the sample are added to the sample plate and the measurement is taken. The refractive index for the sample is recorded.

### Clarity Assessment—Measurement of % Transmittance (% T)

Clarity can be measured by % Transmittance (% T) using Ultra-Violet/Visible (UV/VI) spectrophotometry which determines the transmission of UV/VIS light through a sample. A light wavelength of 600 nm has been shown to be adequate for characterizing the degree of light transmittance through a sample. Typically, it is best to follow the specific instructions relating to the specific spectrophotometer being used. In general, the procedure for measuring percent transmittance starts by setting the spectrophotometer to 600 nm. Then a calibration “blank” is run to calibrate the readout to 100 percent transmittance. A single test sample is then placed in a cuvette designed to fit the specific spectrophotometer and care is taken to ensure no air bubbles are within the sample before the % T is measured by the spectrophotometer at 600 nm. An example of equipment used for measurement is X-Rite Ci7800 Bench Top Spectrophotometer. The compositions of the present invention may have a percent transmittance (% T) of at least about 80% transmittance at 600 nm.

### Combinations

A. A deodorant composition comprising:
   a. a lipophilic carboxylic acid, wherein the lipophilic carboxylic acid has a C Log D from −0.5 to 3 at a pH from 3 to 5; and
b. at least about 20%, by weight of the composition, of a short-chain glycol or polyether compound;
   wherein the composition is free of aluminum.
B. The composition according to paragraph A, wherein the short-chain glycol is dipropylene glycol or propylene glycol.
C. The composition according to paragraphs A and B, further comprising an antimicrobial selected from the group consisting of hexamidine, thymol, polyvinyl formate, niacinamide, cinnamon essential oil, cinnamon bark essential oil, cinnamic aldehyde, piroctone olamine, octenidine dihydrochloride, polyquaternium, and combinations thereof.
D. The composition according to paragraphs A to C, wherein the lipophilic carboxylic acid's total number of carbons is from C7 to C11.
E. The composition according to paragraphs A to D, wherein the composition of free of water.
F. The composition according to paragraphs A to E, wherein the composition is free of citric acid.
G. The composition according to paragraphs A to F, wherein the composition comprises piroctone olamine.
H. The composition according to paragraphs A to G, wherein the composition comprises octenidine dihydrochloride.
I. The composition according to paragraphs A to H, wherein the lipophilic carboxylic acid is an alpha hydroxy acid.
J. The composition according to paragraphs A to I, wherein the alpha hydroxy acid is mandelic acid or hydroxycapric acid.
K. The composition according to paragraphs A to H, wherein the lipophilic carboxylic acid is a dicarboxylic acid.
L. The composition according to paragraphs A to H and K, wherein the dicarboxylic acid is azelaic acid.
M. The composition according to paragraphs A to H and K, wherein the lipophilic carboxylic acid is selected from the group consisting of mandelic acid, hydroxycapric acid, azelaic acid, salicylic acid, or mixtures thereof.
N. The composition according to paragraphs A to M, wherein the composition is in the form of a water in silicone emulsion.
O. The composition according to paragraphs A to N, wherein the composition is a clear gel.
P. The composition according paragraphs A to 0, wherein the composition comprises a water phase and a silicone phase.
Q. The composition according to paragraphs A to P, wherein the refractive index of the water phase is the same as the silicone phase.
R. The composition according to paragraphs A to Q, wherein the composition has a percent transmittance (% T) of at least about 80% transmittance at 600 nm.
S. The composition according to paragraphs A to R, wherein the composition is a water in oil emulsion.
T. The composition according to paragraphs A to S, wherein the refractive index of the water phase is between 1.3500 and 1.4300.
U. The composition according to paragraphs A to T, wherein the composition comprises at least 3% ethanol, by weight of the composition.
V. The composition according to paragraphs A to U, wherein the composition comprises at least of an ionic salt, by weight of the composition.
W. The composition according to paragraph V, wherein the ionic salt is sodium chloride.
X. A deodorant composition comprising:
   a. a lipophilic carboxylic acid, wherein the lipophilic carboxylic acid has a C Log D from −0.5 to 3 at a pH from 3 to 5.
   b. an antimicrobial selected from the group consisting of hexamidine, thymol, polyvinyl formate, niacinamide, cinnamon essential oil, cinnamon bark essential oil, cinnamic aldehyde, piroctone olamine, octenidine dihydrochloride, polyquaternium, and combinations thereof; and
   wherein the composition is free of aluminum.
Y. The composition according to paragraph X, wherein the composition is free of a short-chain glycol.
Z. The composition according to paragraphs X and Y, wherein the composition is free of water.
AA. The composition according to paragraphs X to Z, wherein the lipophilic carboxylic acid is an alpha hydroxy acid.
BB. The composition according to paragraphs X to AA, wherein the alpha hydroxy acid is mandelic acid or hydroxycapric acid.
CC. The composition according to paragraphs X to BB, wherein the lipophilic carboxylic acid is a dicarboxylic acid.
DD. The composition according to paragraphs X to CC, wherein the dicarboxylic acid is azelaic acid.
EE. The composition according to paragraphs X to DD, wherein the composition is free of citric acid.
FF. The composition according to paragraphs X to EE, wherein the composition comprises piroctone olamine.
GG. The composition according to paragraphs X to FF, wherein the composition comprises octenidine dihydrochloride.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about mm”. All numeric values (e.g., dimensions, flow rates, pressures, concentrations, etc.) recited herein may be modified by the term “about”, even if not expressly so stated with the numeric value.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A deodorant composition comprising:
a. a lipophilic carboxylic acid, wherein the lipophilic carboxylic acid has a C Log D from −0.5 to 3 at a pH from 3 to 5; and
b. at least about 20%, by weight of the composition, of a short-chain glycol or polyether compound;
wherein the composition is free of aluminum.

2. The composition of claim 1, wherein the short-chain glycol is dipropylene glycol or propylene glycol.

3. The composition of claim 1, further comprising an antimicrobial selected from the group consisting of hexamidine, thymol, polyvinyl formate, niacinamide, cinnamon essential oil, cinnamon bark essential oil, cinnamic aldehyde, piroctone olamine, octenidine dihydrochloride, polyquaternium, and combinations thereof.

4. The composition of claim 1, wherein the lipophilic carboxylic acid's total number of carbons is from C7 to C11.

5. The composition of claim 1, wherein the composition of free of water.

6. The composition of claim 1, wherein the composition comprises piroctone olamine.

7. The composition of claim 1, wherein the composition comprises octenidine dihydrochloride.

8. The composition of claim 1, wherein the lipophilic carboxylic acid is an alpha hydroxy acid.

9. The composition of claim 1, wherein the lipophilic carboxylic acid is selected from the group consisting of mandelic acid, hydroxycapric acid, azelaic acid, salicylic acid, or mixtures thereof.

10. The composition of claim 1, wherein the composition is in the form of a water in silicone emulsion.

11. The composition of claim 1, wherein the composition is a clear gel.

12. The composition of claim 11, wherein the refractive index of the water phase is the same as the silicone phase.

13. The composition of claim 11, wherein the composition has a percent transmittance (% T) of at least about 80% transmittance at 600 nm.

14. The composition of claim 1, wherein the composition is a water in oil emulsion.

15. The composition of claim 14, wherein the refractive index of the water phase is between 1.3500 and 1.4300.

16. A deodorant composition comprising:
a. a lipophilic carboxylic acid, wherein the lipophilic carboxylic acid has a C Log D from −0.5 to 3 at a pH from 3 to 5.
b. an antimicrobial selected from the group consisting of hexamidine, thymol, polyvinyl formate, niacinamide, cinnamon essential oil, cinnamon bark essential oil, cinnamic aldehyde, piroctone olamine, octenidine dihydrochloride, polyquaternium, and combinations thereof;
and wherein the composition is free of aluminum.

17. The composition of claim 16, wherein the composition is free of a short-chain glycol.

18. The composition of claim 16, wherein the composition is free of water.

19. The composition of claim 16, wherein the lipophilic carboxylic acid is an alpha hydroxy acid selected from mandelic acid or hydroxycapric acid.

20. The composition of claim 16, wherein the composition comprises piroctone olamine, octenidine dihydrochloride, or niacinamide.
